(19) United States
(12) Patent Application Publication
(10) Pub. No.: US 2026/0074013 A1
(43) Pub. Date: Mar. 12, 2026

(54) METHOD AND SYSTEM FOR DETECTING MUTATIONAL SIGNATURES AND THEIR EXPOSURES

(71) Applicant: Ramot at Tel-Aviv University Ltd., Tel-Aviv (IL)

(72) Inventors: Roded SHARAN, Tel-Aviv (IL); Itay SASON, Tel-Aviv (IL); Mark LEISERSON, Tel-Aviv (IL); Yuexi CHEN, Tel-Aviv (IL)

(73) Assignee: Ramot at Tel-Aviv University Ltd., Tel-Aviv (IL)

(21) Appl. No.: 17/971,706

(22) Filed: Oct. 24, 2022

Related U.S. Application Data
(63) Continuation-in-part of application No. PCT/IL2021/050462, filed on Apr. 22, 2021.
(60) Provisional application No. 63/013,571, filed on Apr. 22, 2020.

(51) Int. Cl. G16B 20/20; G16B 30/00; G16B 40/20

(52) U.S. Cl. CPC G16B 20/20; G16B 30/00; G16B 40/20

(57) ABSTRACT

A method of detecting mutational signatures of a sample and their exposures in a collection of samples, each being characterized by nucleic acid sequencing information describing at least one mutation, comprises: clustering the samples to provide clusters and respective exposure vectors, where each exposure vector describes prior probabilities for a plurality of signatures to emit a mutation. An optimization procedure is applied to dynamically re-cluster the samples and to dynamically update the signatures and exposure vectors. Optionally, the mutational signatures in the sample are determined using an exposure vector of one or more clusters associated with the sample.

### RELATED APPLICATIONS

This application is a Continuation-in-Part (CIP) of PCT Patent Application No. PCT/IL2021/050462 having international filing date of Apr. 22, 2021 which claims the benefit of priority under 35 USC § 119 (e) of U.S. Provisional Patent Application No. 63/013,571 filed on Apr. 22, 2020. The contents of the above applications are all incorporated by reference as if fully set forth herein in their entirety.

### FIELD AND BACKGROUND OF THE INVENTION

The present invention, in some embodiments thereof, relates to bioinformatics and, more particularly, but not exclusively, to a method and system for detecting mutational signatures and their exposures.

Cancer genomes are formed by processes that introduce mutations [Thomas et al., Nature Reviews Genetics 15 (9), 585-598 (2014), doi: 10.1038/nrg3729; Anthony et al., Cell 168 (4), 644-656 (2017), doi: 10.1016/j.cell.2017.01.002]. Mutation signatures have been linked to exposure to specific carcinogens, such as tobacco smoke and ultraviolet radiation [Ludmil et al., Nature 500 (7463), 415-421 (2013), doi: 10.1038/nature12477; Ludmil et al., Science 354 (6312), 618-622 (2016), doi: 10.1126/science.aag0299].

A recent study [Helen et al., Nature Medicine 23 (4), 517-525 (2017), doi: 10.1038/nm.4292] estimated an increase in the number of breast cancer patients with homologous recombination repair deficiency when using mutational signatures compared to other approaches. Statistical models for discovering and characterizing mutational signatures are known, and are typically applicable for whole-genome or whole-exome sequencing. Known in the art is a method in which non-negative matrix factorization (NMF) is used to discover mutation signatures [see, e.g., Ludmil, Nature 500, supra, and Ludmil et al. Cell Reports 3 (1), 246-259 (2013), doi: 10.1016/j.celrep.2012.12. 008]. Subsequent methods have used different forms of NMF [Kyle et al., bioRxiv, 036541 (2016), doi: 10.1101/036541; Andrej et al., Genome Biology 14 (4), 1-10 (2013), doi: 10.1186/gb-2013-14-4-r39; Jacgil et al., Nature Genetics 48 (6), 600-606 (2016), doi: 10.1038/ng.3557; Rafael et al., Bioinformatics 33 (1), 8-16 (2016), doi: 10.1093/bioinformatics/btw572], or focused on inferring the exposures (also known as refitting) given the signatures and mutation counts [Xiaoqing et al., Bioinformatics (Oxford and England) (2017), doi: 10.1093/bioinformatics/btx604; Rachel et al., Biology 17 (1), 31 (2016), doi: 10.1186/s13059-016-0893-4; Blokzijl et al., Genome Medicine 10, 33 (2018); doi: 10.1186/s13073-018-0539-0]. Another class of approaches borrows from the world of topic modeling, aiming to provide a probabilistic model of the data so as to maximize the model's likelihood [Funnell et al., PLOS Computational Biology 15 (2): €1006799 (2019); Yuichi et al., PLOS Genetics 11 (12), 1005657 (2015). doi: 10.1371/journal.pgen.1005657; Wojtowicz et al., Genome Medicine 11, 49 (2019). doi: 10.1186/s13073-019-0659-1; Robinson et al., Bioinformatics 35 (14), 492-500 (2019), doi: 10.1093/bioinformatics/btz340].

Additional background art includes Signature Multivariate Analysis (SigMA) which is a method that relies on whole-genome training data to interpret sparse samples and predict their homologous recombination deficiency status [Gulhan et al., Nature Genetics 51, 912-919 (2019), doi: 10.1038/s41588-019-0390-2].

### SUMMARY OF THE INVENTION

According to some embodiments of the invention there is provided a method of detecting mutational signatures of a sample and their exposures in a collection of samples, each being characterized by nucleic acid sequencing information describing at least one mutation. The method comprises: clustering the samples to provide clusters and respective exposure vectors, each exposure vector describing prior probabilities for a plurality of signatures to emit a mutation; and applying an optimization procedure to dynamically re-cluster the samples and to dynamically update the signatures and exposure vectors. The method optionally and preferably comprises determining the mutational signatures in the sample and their exposure vector, based on an output of the optimization procedure, e.g., using an exposure vector of one or more clusters associated with the sample under analysis.

According to some embodiments of the invention, the method comprises inferring mutational signatures each specifying a probability of emitting each of known mutation categories.

According to some embodiments of the invention, the method comprises using known mutational signatures, each specifying a probability of emitting each of known mutation categories.

According to some embodiments of the invention, the clustering is a hard clustering, wherein each sample of the collection belongs to a single cluster.

According to some embodiments of the invention, the clustering is a soft clustering, wherein at least one sample of the collection is associated with at least two clusters characterized by different exposure vectors.

According to some embodiments of the invention the clustering comprises calculating cluster prior probabilities, and wherein the optimization procedure dynamically updates the cluster prior probabilities.

According to some embodiments of the invention the clustering comprises estimating mutational signatures shared among samples, wherein the optimization procedure dynamically updates the shared mutational signatures.

According to some embodiments of the invention the optimization procedure comprises an Expectation-Maximization procedure. According to some embodiments of the invention the optimization procedure comprises at least one of: a gradient descent procedure, a neural network procedure, an evolutionary procedure, and a simulated annealing procedure.

According to some embodiments of the invention, the mutational signatures comprise mutational signatures of homologous recombination deficiencies.

According to some embodiments of the invention the mutations comprise somatic mutations.

According to some embodiments of the invention the mutations comprise cancer mutations.

According to some embodiments of the invention the cancer is selected from the group consisting of pancreatic cancer, breast cancer, and ovarian cancer. According to some embodiments of the invention the cancer is selected from the group consisting of colorectal cancer, esophageal cancer, prostate cancer, renal cancer and liver cancer.

According to some embodiments of the invention, for the sample and for at least a few samples in the collection, the nucleic acid sequencing information describes less than 20 mutations, more preferably less than 15 mutations, more preferably less than 10 mutations.

According to some embodiments of the invention, for the sample and for at least a few samples (e.g., at least 20% or at least 50% or at least 80% of the sample, or each of the samples) in the collection, the nucleic acid sequencing information describes less than 20 mutations, more preferably less than 15 mutations, more preferably less than 10 mutations.

According to an aspect of some embodiments of the present invention there is provided a computer software product, comprising a computer-readable medium in which program instructions are stored, which instructions, when read by a data processor, cause the data processor to receive nucleic acid sequencing information characterizing each sample in a collection of samples, to access a computer readable medium storing known mutational categories, and to execute the method as described and optionally and preferably as exemplified herein.

According to some embodiments of the invention, at least one of the signatures comprises a set of values, each describing a probability associated with a known mutational category.

According to some embodiments of the invention, the known mutational category is one of a group of somatic mutation categories. According to some embodiments of the present invention the group of somatic mutation categories comprises 96 categories.

According to some embodiments of the invention, the known mutational category is one of a group of germline mutation categories.

According to an aspect of some embodiments of the present invention there is provided a system for detecting mutational signatures of a sample in a collection of samples, the system comprising: an input circuit receiving nucleic acid sequencing information characterizing each sample in the collection of samples; a computer readable medium storing known mutational categories; and a data processor configured for executing the method as described and optionally and preferably as exemplified herein.

According to some embodiments of the invention the nucleic acid sequencing information comprises information obtained by targeted sequencing.

According to some embodiments of the invention the method comprises obtaining the nucleic acid sequencing information by sequencing a collection of genome windows obtained from an output of a machine learning procedure trained to identify genome regions that are indicative of an activity of a signature of the at least one mutation.

According to some embodiments of the invention the method comprises feeding a whole genome to the trained machine learning procedure.

According to some embodiments of the invention the trained machine learning procedure is trained to select an optimally-scoring subset of a set of genome windows.

According to some embodiments of the invention the trained machine learning procedure is configured to divide the set into nonoverlapping windows, to compute, with a window scoring function, a measure of mutational signature activity in each window across the collection of samples, and to combine highest scoring windows.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration showing a plate diagram for a Multinomial Mixture Model (MMM);

FIG. 2 is a schematic illustration showing a plate diagram for a mixture of MMMs;

FIGS. 3A-D show performance evaluation on simulated data, as obtained in experiments performed according to some embodiments of the present invention;

FIGS. 4A and 4B show performance evaluation in a clinical setting, as obtained in experiments performed according to some embodiments of the present invention;

FIGS. 5A-E show performance evaluation on MSK-IMPACT data, as obtained in experiments performed according to some embodiments of the present invention;

FIGS. 6A-F show a first de-novo signature from the MSK-IMPACT data, as obtained in experiments performed according to some embodiments of the present invention;

FIGS. 7A-F show a second de-novo signature from the MSK-IMPACT data, as obtained in experiments performed according to some embodiments of the present invention;

FIGS. 8A-F show a third de-novo signature from the MSK-IMPACT data, as obtained in experiments performed according to some embodiments of the present invention;

FIGS. 9A-F show a fourth de-novo signature from the MSK-IMPACT data, as obtained in experiments performed according to some embodiments of the present invention;

FIGS. 10A-F show a fifth de-novo signature from the MSK-IMPACT data, as obtained in experiments performed according to some embodiments of the present invention;

FIGS. 11A-F show a sixth de-novo signature from the MSK-IMPACT data, as obtained in experiments performed according to some embodiments of the present invention;

FIGS. 12A and 12B show clusters learned from the MSK-IMPACT data, as obtained in experiments performed according to some embodiments of the present invention;

FIGS. 13A-D show signature discovery from the MSK-IMPACT data, as obtained in experiments performed according to some embodiments of the present invention;

FIGS. 14A-D shows survival analysis of Mix patient clusters, as obtained in experiments performed according to some embodiments of the present invention;

FIG. 15 is a flowchart diagram of a method suitable for analyzing sequencing data according to various exemplary embodiments of the present invention;

FIG. 16 is a schematic illustration of computing system which can be used according to some embodiments of the present invention for executing the method shown in FIG. 15;

FIGS. 17A-D are schematic illustrations of a method suitable for designing a gene panel, according to some embodiments of the present invention; and

FIG. 18 shows assessment of genomic panels constructed according to some embodiments of the present invention for their ability to predict mutational signature activity at various panel sizes.

### DESCRIPTION OF SPECIFIC EMBODIMENTS OF THE INVENTION

The present invention, in some embodiments thereof, relates to bioinformatics and, more particularly, but not exclusively, to a method and system for detecting mutational signatures and their exposures.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

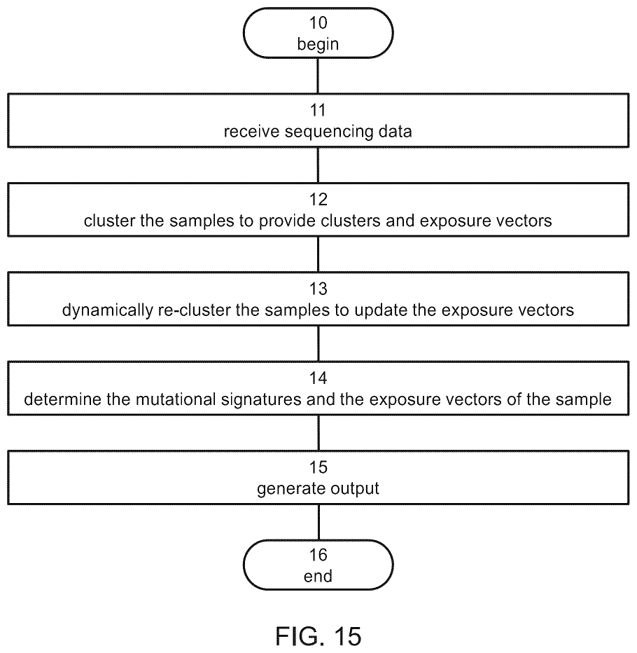

FIG. 15 is a flowchart diagram of a method suitable for analyzing sequencing data of a sample in a collection of samples according to various exemplary embodiments of the present invention. It is to be understood that, unless otherwise defined, the operations described herein below can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

At least part of the operations described herein can be implemented by a data processing system, e.g., a dedicated circuitry or a general purpose computer, configured for receiving data and executing the operations described below. At least part of the operations can be implemented by a cloud-computing facility at a remote location.

Computer programs implementing the method of the present embodiments can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. The computer programs can be run by loading the code instructions either from their distribution medium or their intermediate storage medium into the execution memory of the computer, configuring the computer to act in accordance with the method of this invention. During operation, the computer can store in a memory data structures or values obtained by intermediate calculations and pulls these data structures or values for use in subsequent operation. All these operations are well-known to those skilled in the art of computer systems.

Processing operations described herein may be performed by means of processer circuit, such as a DSP, microcontroller, FPGA, ASIC, etc., or any other conventional and/or dedicated computing system.

The method of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

Each of the samples in the collection of samples is typically characterized by nucleic acid sequencing information describing at least one mutation. Typically, but not necessarily, the mutations include somatic mutations. Also contemplated are embodiments in which the mutations include germline mutations. The number of samples in the collection is denoted N. Typically, the mutation(s) in each sample are categorized into categories, such as, but not limited to, one or more of the 96 known categories for somatic mutations, or one or more of a group of germline mutation categories. The mutations, or mutation categories, of the nth sample (n=1, . . . ,N) are denoted as a vector Oⁿ of length Tₙ, Oⁿ=(o₁ . . . o_{Tn}), where oᵢ (i=1, . . . ,Tₙ) is the ith element of the vector Oⁿ.

In some embodiments, the mutations comprise cancer mutations. Representative examples of cancer types for which the method can be useful, include, without limitation, pancreatic cancer, breast cancer, ovarian cancer. Additional examples include colorectal cancer, esophageal cancer, prostate cancer, renal cancer and liver cancer.

The method of the present embodiments can be used in more than one way. In some embodiments of the present invention the method is used for de-novo signature discovery. In these embodiments, the method typically receives a collection of known mutations, or more preferably a collection of known mutation categories, and provides one or more mutational signatures (such as, but not limited to, mutational signatures of homologous recombination deficiencies). In alternative embodiments of the present invention, the method receives both a collection of known mutations or known mutation categories, and a collection of known mutational signatures (e.g., mutational signatures of homologous recombination deficiencies) and provides their respective exposures.

When the mutations are categorized, one or more of the signatures, e.g., each of the signatures, can comprise a set of values, each describing a probability associated with a known mutational category. Thus, a signature in these embodiments describes a set of probabilities for emitting a respective set of known mutation categories.

The method begins at 10 and optionally and continues to 11 at which nucleic acid sequencing data describing the each of the samples in the collection are received. The sequencing data describing the sample may, in some embodiments, include sparse sequencing data. The data are “sparse” in the sense that there is a relatively small number of mutations in the sample. In various exemplary embodiments of the invention the sequencing data describing at least a few, or each, of the other samples in the collection are also sparse. As a representative example, which is not to be considered as limited, the sequencing data describing the sample, and optionally and preferably also the sequencing data describing at least a few of the samples (e.g., at least 20% or at least 50% or at least 80% of the samples, or each of the samples) in the collection, includes less than X mutations, where X is equal to 20, or 18, or 16, or 14, or 12, or 10, or 8, or 6 or 4.

The advantage of having sparse sequencing data is that it allows the method of the present embodiments to be applied also to data obtained in targeted (gene panel) sequencing assays, and does not have to rely on whole-genome sequencing (WGS) or even whole-exome sequencing (WXS).

The gene panel can be a focused gene panel selected for a preselected set of genes or gene regions associated with a particular disease or phenotype. The gene panel can alternatively be optimized for detection of a preselected mutational signature by sequencing a collection of genome windows, preferably nonoverlapping genome windows. The genome windows may in some embodiments of the present invention be of a fixed and predetermined width. Such a collection can optionally and preferably be obtained by feeding the whole genome to a trained machine learning procedure, and receiving the collection of genomic segments from the output layer of the trained machine learning procedure. The machine learning procedure can be trained to identify genome regions that are indicative of an activity of the the preselected mutational signature.

In some embodiments of the present invention the machine learning procedure is trained using a training set of samples that includes a subset in which the preselected signature is active. Once trained, the machine learning selects an optimally-scoring subset from the set of all nonoverlapping genome windows. A representative example of a machine learning procedure suitable for the present embodiments, and of a preferred process training for training such a machine learning procedure is provided in the Examples section that follows.

The method optionally and preferably proceeds to 12 at which the samples in the collection are clustered to provide a plurality of clusters and a respective plurality of exposure vectors.

The number of clusters is denoted by L, and the exposure vector of the [symbol] th cluster ( [symbol] =1, . . . ,L) is denoted π [symbol] . Thus, each cluster is associated with an exposure vector. This is different from conventional Multinomial Mixture Model (MMM), in which the exposure vectors are associated with individual samples (one exposure vector per sample). The elements of the exposure vector π [symbol] of the [symbol] th cluster represent prior probabilities for a respective plurality of signatures to emit a mutation. In some embodiments the clustering comprises calculating cluster prior probabilities, and in some embodiments the clustering comprises estimating mutational signatures shared among samples.

The clustering 12 can be a hard clustering, wherein each sample of the collection belongs to a single cluster, or a soft clustering, wherein at least one sample of the collection is associated with two or more clusters characterized by different exposure vectors. For example, consider the nth sample. In hard clustering, the exposures can be defined based on the most likely cluster for a given sample. In soft clustering a sum, more preferably a weighted sum, of all the clusters' exposures can be calculated. In some embodiments, both hard and soft clustering are employed. Preferably, hard-clustering is executed to cluster the samples, and soft clustering is executed to obtain the exposures.

At 13 the method and applies an optimization procedure to dynamically re-cluster the samples, and to dynamically update the signatures and the exposure vectors. Optionally and preferably, aside for the exposure vectors, the optimization dynamically updates also the cluster prior probabilities and/or the mutational signatures that are shared among samples.

The optimization is typically executed to maximize a likelihood function, which expresses the probability to have a set V of N mutation occurrence vectors, or more preferably of N mutation category occurrence vectors, for a set π of L exposure vectors and optionally and preferably a set w of L cluster prior probabilities and/or a set e of shared mutational signatures. The nth element of the set V is a vector that corresponds to the nth sample and that typically includes the number of times that each of the mutation or mutation category appears in that sample. Thus, for example, suppose, for simplicity that a particular sample contains only the first mutation category. The first vector in the set V that corresponds to this particular sample has zero elements for each mutation category other than the first mutation category, and a one non-zero element describing the number of occurrences of the first mutation category in it.

The optimization procedure is typically re-executed, e.g., iteratively, until a predetermined stopping criterion or set of stopping criteria is met. The stopping criteria can include a criterion pertaining to the number of iterations, and/or a criterion pertaining to the convergence of the likelihood function. Thus, for example, at each execution of the optimization procedure, the method can compare the current value of the likelihood function to its previous value and terminate the execution when the two values are sufficiently close (e.g., when their difference or ratio is below a predetermined threshold), or when the total number of executions is above a predetermined threshold.

The preferred optimization procedure is Expectation-Maximization (EM) procedure, but other optimization procedures, or combinations of optimization procedures are also contemplated. Representative examples of optimization procedures suitable for the present embodiments include, without limitation, a gradient descent procedure, a neural network procedure, an evolutionary procedure, and a simulated annealing procedure. When an EM procedure is employed, the procedure alternate between performing an expectation step (referred to as the “E-step”), in which the expectation of the likelihood is calculated using the current estimate for the parameters π, w and e, and a maximization step (referred to as the “M-step”), in which parameters that maximize the expected likelihood found on the E-step are calculated. A Preferred example of an EM process suitable for the present embodiments is described in the Examples section that follows.

When the method receives a collection of known signatures, the update step of the shared mutation signature e can be skipped and the initial value for it can be set to the known signatures.

Once the optimization procedure is completed, a set of L clusters and a corresponding set of L exposure vectors is obtained. The method can then optionally continue to 14 at which the mutational signatures in the sample, and optionally and preferably also the exposure vector of the mutational signatures in the sample, is determined based on the output of the optimization procedure. The determination at 14 preferably uses the obtained clusters and their exposure vectors. The method can determine the mutational signatures in the sample using an exposure vector of at least one cluster associated with the sample. When the method receives a collection of known mutations or known mutation categories, as well as a collection of known mutational signatures, operation 14 can be skipped.

The method proceeds to 15 at which an output pertaining to the determined mutation signatures and/or the exposure vectors is generated. When the mutational signatures are known, the output optionally and preferably includes the known mutation signatures and the obtained exposure vectors. When the mutational signatures are determined at 14, the output optionally and preferably includes the determined mutation signatures and the respective exposure vectors. The output can be displayed or transmitted to a remote location for display at the remote location, or stored in a computer readable medium.

The method ends at 16.

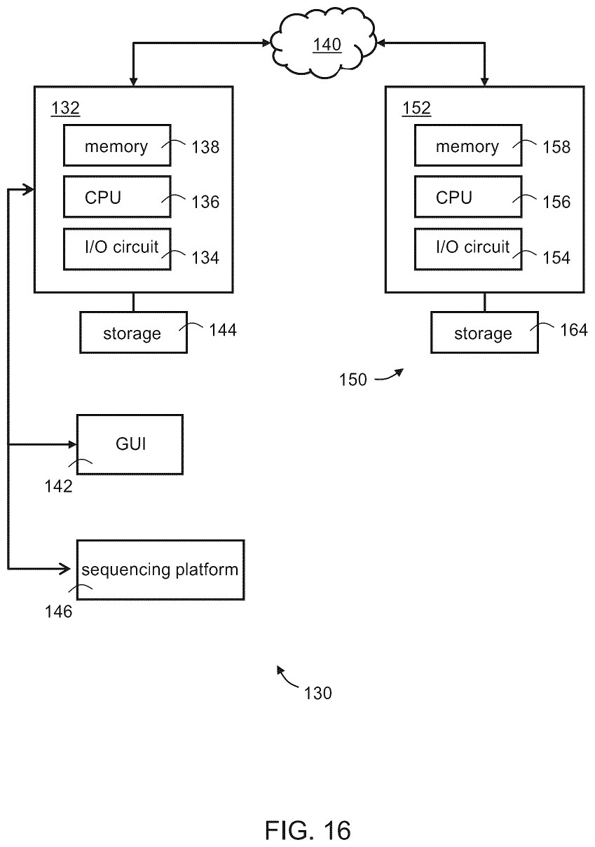

FIG. 16 is a schematic illustration of a client computer 130 having a hardware processor 132, which typically comprises an input/output (I/O) circuit 134, a hardware central processing unit (CPU) 136 (e.g., a hardware microprocessor), and a hardware memory 138 which typically includes both volatile memory and non-volatile memory. CPU 136 is in communication with I/O circuit 134 and memory 138. Client computer 130 preferably comprises a graphical user interface (GUI) 142 in communication with processor 132. I/O circuit 134 preferably communicates information in appropriately structured form to and from GUI 142. Also shown is a server computer 150 which can similarly include a hardware processor 152, an I/O circuit 154, a hardware CPU 156, a hardware memory 158. I/O circuits 134 and 154 of client 130 and server 150 computers can operate as transceivers that communicate information with each other via a wired or wireless communication. For example, client 130 and server 150 computers can communicate via a network 140, such as a local area network (LAN), a wide area network (WAN) or the Internet. Server computer 150 can be in some embodiments be a part of a cloud computing resource of a cloud computing facility in communication with client computer 130 over the network 140. Further shown, is a sequencing platform 146 that is associated with client computer 130, and that provides sequencing data describing the sample(s), e.g., by performing a targeted sequencing assay as known in the art.

GUI 142 and processor 132 can be integrated together within the same housing or they can be separate units communicating with each other. Similarly, platform 146 and processor 132 can be integrated together within the same housing or they can be separate units communicating with each other.

GUI 142 can optionally and preferably be part of a system including a dedicated CPU and I/O circuits (not shown) to allow GUI 142 to communicate with processor 132. Processor 132 issues to GUI 142 graphical and textual output generated by CPU 136. Processor 132 also receives from GUI 142 signals pertaining to control commands generated by GUI 142 in response to user input. GUI 142 can be of any type known in the art, such as, but not limited to, a keyboard and a display, a touch screen, and the like.

Client 130 and server 150 computers can further comprise one or more computer-readable storage media 144, 164, respectively. Media 144 and 164 are preferably non-transitory storage media storing computer code instructions for executing the method as further detailed herein, and processors 132 and 152 execute these code instructions. The code instructions can be run by loading the respective code instructions into the respective execution memories 138 and 158 of the respective processors 132 and 152.

Each of storage media 144 and 164 can store program instructions which, when read by the respective processor, cause the processor to receive the sequencing data from platform 146 clustering the samples, apply an optimization procedure, and determine the mutational signatures in the sample (when not provided as input) and/or exposure vectors as further detailed hereinabove.

In some embodiments of the present invention, sequencing information is generated as digital data by platform 146 which are transmitted to processor 132 by means of I/O circuit 134. Processor 132 receives the digital sequencing data, and analyzes the data as further detailed hereinabove. Computer 130 can display the mutational signatures and/or exposure vectors on GUI 142, or store them in storage medium 144. Alternatively, processor 132 can transmit the digital sequencing data over network 140 to server computer 150. Computer 150 receives the digital sequencing data, analyzes the data as further detailed hereinabove, and transmits mutational signatures in the sample and/or exposure vectors back to computer 130 over network 140. Computer 130 receives the mutational signatures and/or exposure vectors and displays them on GUI 142 or stores them in storage medium 144.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1: Signature Discovery from Sparse Data

Each cancer genome is shaped by a combination of processes that introduce mutations over time [1, 2]. The incidence and etiology of these mutational processes may provide insights into tumorigenesis and personalized therapy. It is thus beneficial to uncover the characteristic signatures of active mutational processes in patients from their patterns of single base substitutions. Some such mutation signatures have been linked to exposure to specific carcinogens, such as tobacco smoke [6] and ultraviolet radiation [3]. Other mutation signatures arise from deficient DNA damage repair pathways. By serving as a proxy for the functional status of the repair pathway, mutational signatures provide an avenue around traditional driver mutation analyses. This is useful for personalizing cancer therapies, many of which work by causing DNA damage or inhibiting DNA damage response or repair genes [7, 8, 9, 10], because the functional effect of many variants is hard to predict.

Indeed, a recent study [11] estimated a >4-fold increase in the number of breast cancer patients with homologous recombination repair deficiency-making them eligible for PARP inhibitors [12]—when using mutational signatures compared to current approaches. Thus, understanding the signatures of mutational processes may lead to the development of many effective diagnostic and treatment strategies.

Statistical models for discovering and characterizing mutational signatures are used for realizing their potential as biomarkers in the clinic. A broad catalogue of mutational signatures in cancer genomes was only recently revealed through computational analysis of mutations in thousands of tumors. Alexandrov et al. [3, 4] were the first to use non-negative matrix factorization (NMF) to discover mutation signatures. Subsequent methods have used different forms of NMF [13, 14, 15, 16], or focused on inferring the exposures (also known as refitting) given the signatures and mutation counts [17, 18, 19].

A more recent class of approaches borrows from the world of topic modeling, aiming to provide a probabilistic model of the data so as to maximize the model's likelihood [20, 21, 22, 23].

These previous methods are applicable for whole-genome or even whole-exome sequencing. However, they cannot handle very sparse data as obtained routinely in targeted (gene panel) sequencing assays. There is only a single method, SigMA [Gulhan et al., Nature Genetics 51, 912-919 (2019), doi: 10.1038/s41588-019-0390-2], that attempts to address this challenge by relying on whole-genome training data to interpret sparse samples and predict their homologous recombination deficiency status. However, SigMA still suffers from the fact that not all cancer types have available whole-genome sequencing data.

This Example presents a technique that handles sparse targeted sequencing data without pre-training on rich data. The model of the present embodiments simultaneously clusters the samples and learns the mutational landscape of each cluster, thereby overcoming the sparsity problem. Using synthetic and real targeted sequencing data, this Example shows that the technique of the present embodiments is superior to current non-sparse approaches in signature discovery, signature refitting and patient stratification. This Example demonstrates the utility of the model of the present embodiments in several clinical settings.

### Methods

### Preliminaries

Somatic mutations in cancer are assumed to fall into M=96 categories (denoting the mutation identity and its flanking bases). These mutations are assumed to be the result of the activity of K (a hyper-parameter) mutational processes, each of which is associated with a signature Sᵢ=(eᵢ(1) . . . eᵢ(M)) of probabilities to emit each of the mutation categories. The mutation categories observed in a given tumor n is denoted by Oⁿ=(o₁ . . . o_{Tn}). This sequence is assumed to have been emitted by the (hidden) signature sequence Zⁿ=(z₁ . . . z_{Tn}).

### Multinomial Mixture Model (MMM)

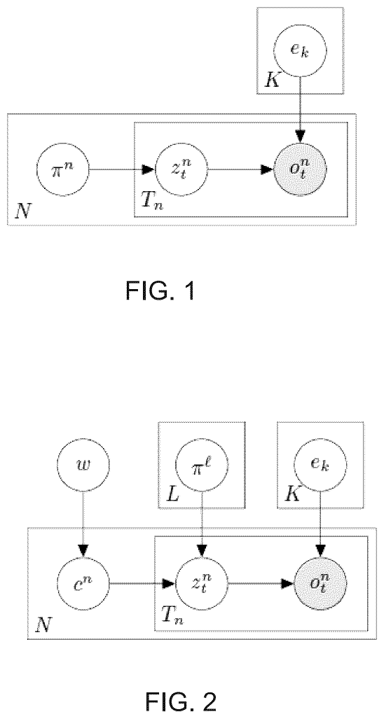

The basic multinomial mixture model is depicted in FIG. 1. The model is parameterized by the signatures S₁, . . . , S_{K} and their exposure vector π, where πᵢ is the prior probability for the ith signature to emit any given mutation. In the following it is assumed, for simplicity, that a single sample facilitates the generalization to the model presented herein. Given the observed mutations O and the unobserved signatures Z, the model's likelihood is:

$$Pr [ O ] = {\prod }_{t = 1}^{T} PR [ {o}_{t} ] = {\prod }_{t = 1}^{T} {\sum }_{i = 1}^{K} Pr [ {o}_{t} , {z}_{t} = i ] = {\prod }_{t = 1}^{T} {\sum }_{i = 1}^{K} {π}_{i} {e}_{i} ( {o}_{t} )$$

Denoting by Vⱼ=|{t|oₜ=j}| the number of times the jth category appears in the data, the likelihood can be rewritten as:

$$f ( V , π , e ) := {\prod }_{j = 1}^{M} {( {\sum }_{i = 1}^{K} {π}_{i} {e}_{i} ( j ) )}^{{V}_{j}}$$

The likelihood can be maximized using the Expectation Maximization (EM) algorithm. In the E-step the method of the present embodiments computes the expectation of the model's emissions and (relative) exposures under the current assignment to those parameters. Specially, the expected number of times that signature i emitted mutation category j is computed by

$${E}_{i} ( j , V , π , e ) := \frac{{V}_{j} {π}_{i} {e}_{i} ( j )}{{\sum }_{k = 1}^{K} {π}_{k} {e}_{k} ( j )}$$

and the expected number of times signature i was used is computed by

$${A}_{i} ( V , π , e ) := {\sum }_{j = 1}^{M} {E}_{i} ( j , V , π , e ) .$$

These expectations are normalized (to probabilities) in the M-step to yield a new set of parameters until convergence.

It is appreciated that in this model, given a collection of samples, one cannot expect all of them to have the same exposures x. While it is possible to learn a unique exposure vector per sample, as done by conventional methods, the number of parameters then grows linearly with the number of samples, which may lead to overfitting in a sparse data scenario.

### Mix: a Mixture of MMMs

According to some embodiments of the present invention the samples are clustered and the exposures per cluster are learned (rather than exposures per sample).

To this end, the Inventors use a mixture model and a scheme to optimize its likelihood, leading to simultaneous optimization of sample (soft) clustering, exposures and signatures (FIG. 2). Given a hyper-parameter L indicating the number of clusters, denote by cⁿ ∈{1 . . . L} the hidden variables representing the true cluster identity of each sample.

According to some embodiments of the present invention one or more, more preferably each of the following quantities are learned: cluster prior probabilities w=(w₁ . . . w_{L}), cluster exposures π=(π₁ . . . π_{L}), and shared signatures e, so as to maximize the model's likelihood. The likelihood is optionally and preferably:

$$\begin{matrix}Pr [ V \vert w , π , e ] = {\prod }_{n = 1}^{N} Pr [ {V}^{n} \vert w , π , e ] \\ = {\prod }_{n = 1}^{N} {\sum }_{ℓ = 1}^{L} Pr [ {c}^{n} = ℓ , {V}^{n} \vert w , π , e ] \\ = {\prod }_{n = 1}^{N} {\sum }_{ℓ = 1}^{L} Pr [ {c}^{n} = ℓ ] Pr [ {V}^{n} \vert {π}^{ℓ} , e ] \\ = {\prod }_{n = 1}^{N} {\sum }_{ℓ = 1}^{L} {w}_{ℓ} f [ {V}^{n} , {π}^{ℓ} , e ]\end{matrix}$$

A preferred optimization process is by Expectation-Maximization (EM) iterative process alternating between performing an expectation step (E-step), in which the expectation of the likelihood is calculated using the current estimate for the parameters, and a maximization step (M-step), in which parameters that maximize the expected likelihood found on the E-step are calculated. A Preferred example of an EM process suitable for the present embodiments is:

E-step: Compute for every i, j, n, [symbol] :

$${f}^{n , ℓ} = Pr [ {c}^{n} = ℓ \vert {V}^{n} , w , π , e ] = \frac{{w}_{ℓ} f ( {V}^{n} , {π}^{ℓ} , e )}{{\sum }_{{ℓ}^{′} = 1}^{L} {w}_{{ℓ}^{′}} f ( {V}^{n} , {π}^{{ℓ}^{′}} , e )} \\ {E}_{i} ( j ) = {\sum }_{n = 1}^{N} {\sum }_{ℓ = 1}^{L} {f}^{n , ℓ} {E}_{i} ( j , {V}^{n} , {π}^{ℓ} , e ) \\ {A}_{i}^{ℓ} = {\sum }_{n = 1}^{N} {f}^{n , ℓ} {A}_{i} ( {V}^{n} , {π}^{ℓ} , e ) \\ {W}_{ℓ} = {\sum }_{n = 1}^{N} {f}^{n , ℓ}$$

M-step: Compute for every i, j, [symbol] :

$${π}_{i}^{ℓ} = \frac{{A}_{i}^{ℓ}}{{\sum }_{{i}^{′} = 1}^{K} {A}_{{i}^{′}}^{ℓ}} \\ {e}_{i} ( j ) = \frac{{E}_{i} ( j )}{{\sum }_{{j}^{′} = 1}^{M} {E}_{i} ( {j}^{′} )} \\ {w}_{ℓ} = \frac{{W}_{ℓ}}{{\sum }_{{ℓ}^{′} = 1}^{L} {W}_{{ℓ}^{′}}}$$

A detailed derivation of the algorithm is given in the Annex. To learn the model in a refitting setting, with fixed known signatures, the update step of e can be skipped and the initial value for it can be set to the given signatures. Each EM iteration can be completed in O(NLK) time for N samples, L clusters and K signatures. The EM algorithm is optionally and preferably executed until it converges to a local maximum and up to a predetermined number (e.g., from about 500 to about 2,000, for example, about 1,000) of iterations. Preferably, the model is trained several times (e.g., from 5 to 20 times, for example, 10 times) with different random seeds, and the output that yields the highest likelihood is selected. The advantage of this embodiment is that it avoids being trapped in poor local maxima,

To estimate the hyper parameters of Mix (L, K) the Bayesian information criterion (BIC) was used to weigh the tradeoff between model fit and the number of parameters. The model was trained on a range of choices for L and K. The hyper parameters were chosen to be:

$$L * , K *= {arg min}_{L , K} \{ Mix . size \cdot log ( n ) - 2 log ( Mix . prob ) \}$$

where Mix.size is the number of parameters in the model, n is the number of data points (number of mutations) and Mix.prob is the probability of the data given the trained model. The total number of learned parameters in Mix is given by (L−1)+L (K−1)+K (M−1), where M is the number of mutation categories.

Given a trained model [w, π, e] and a sample V an exposure vector E is constructed for it. This Example explores two inference schemes, referred to as Hard clustering, and Soft clustering.

In the hard clustering scheme, the exposures are optionally and preferably defined based on the most likely cluster for that sample. In this case E=π [symbol] where [symbol] is the cluster that maximizes

$${f}^{ℓ} := Pr [ {c}^{n} = ℓ \vert V , w , π , e ] .$$

In the Soft clustering scheme, a weighted sum of all clusters' exposures is optionally and preferably calculated, with f [symbol] as weights:

$$E = {\sum }_{ℓ = 1}^{L} {f}^{ℓ} {π}^{ℓ}$$

In both schemes E is the normalized exposure, and is therefore summed to 1. In some embodiments of the present invention so the normalized exposure E is multiplied by the number of mutations to obtain the real exposures. For some applications, however, it may be desired to use the normalized exposures as such, without multiplying them by the number of mutations. Preferably, hard-clustering is typically used to cluster the samples, and soft clustering is typically used to obtain the exposures.

This Example presents both de-novo experiments, in which mutational signatures are learned, and refitting experiments, in which the signatures are assumed to be given. In the latter cases, the analyses is described for Single Base Substitution (SBS) mutation signatures in COSMIC [www(dot)cancer(dot)sanger(dot)ac(dot)uk /cosmic/signatures_v2(dot)tt] that are known to be active in the cancer type being analyzed.

### Mutation and Clinical Data

Mix was applied to analyze mutational signatures in three datasets.

### Somatic Mutation Data

MSK-IMPACT [26, 27] Pan-Cancer. mutations were downloaded for a cohort of patients with Memorial Sloan Kettering Integrated Mutation Profiling of Actionable Cancer Targets (MSK-IMPACT) targeted sequencing data from www(dot)cbioportal(dot)org/The MSK-IMPACT dataset contains 11,369 pan-cancer patients' sequencing samples across 410 target genes. The analysis was applied to the 18 cancer types with more than 100 samples, which results in a dataset of 5931 samples and an average of 6.8 mutations per sample. According to COSMIC there are 17 mutational signatures that are active in those cancer types, 12 of which are associated with more than 5% of the mutations. The 17 active COSMIC signatures are Signatures 1-8, Signatures 10-13, Signatures 15-17, Signature 20 and Signature 21.

ICGC breast cancers (BRCA). Mutations for 560 breast cancer patients were downloaded with whole-genome sequencing data from the International Cancer Genome Consortium [28]. There are about 6214 mutations per sample in this collection and 12 active COSMIC signatures are associated with it. The 12 active COSMIC signatures in breast cancer are 1, 2, 3, 5, 6, 8, 13, 17, 18, 20, 26 and 30.

TCGA ovarian cancers (OV). Mutations from whole-exome sequencing data of 411 ovarian cancer patients were downloaded from the Cancer Genome Atlas [29]. There are about 113 mutations per sample in this collection and 3 active signatures are associated with it. The 3 active COSMIC signatures are 1, 3 and 5.

Additional analysis was applied to mutation data sets for which clinical information exists on homologous recombination deficiency (HRD) status or immunotherapy response.

### Clinically-Oriented Data

Whole Genome Sequencing of Triple Negative breast cancers. Triple negative whole genome breast cancers data along with their HRDetect-predicted labels from Staaf et al. [30]. The output labels are categorized by the probability of HRD: high (HRD score above 0.7), intermediate (0.2 to 0.7), and low (below 0.2). Overall, 139 patients are predicted as “high”, 13 are predicted as “intermediate”, and 85 are predicted as “low”. To make the labels binary the 13 “intermediate” labeled samples were removed, leaving 224 samples, 62% of which with HRD.

MSK-IMPACT sequencing of Non-small cell lung cancer (NSCLC) data treated by CTLA-4/PD-L1 [31]. Data were downloaded from the cBioPortal [32, 33]. There are 240 NSCLC patients in this cohort. 206 patients went through PD-L1 monotherapy, and 34 patients went through a combined therapy of PD-L1 and CTLA-4. To have a clean dataset to analyze, the 150 LUAD patients that were treated with PD-L1 monotherapy and either showed durable clinical benefit (41 samples) or not (109 samples) were used.

MSK-IMPACT sequencing of pan-cancer patients data treated by CTLA4, PD-1, and/or PD-L1 [34]. Mutation profiles of pan-cancer patients with survival information were downloaded from the cBioPortal [32, 33] 1583. In detail, there are 339 non-small cell lung cancers, 311 melanoma, 209 bladder cancers, 137 renal cell carcinoma, 126 head and neck cancers, 115 esophagogastric cancers, 114 gliomas, 109 colorectal cancers, 83 cancers of unknown primary, 39 breast cancers, and 1 skin cancer (non-melanoma). 1243 of the patients were treated with PD-1/PD-L1, 95 were treated with CTLA4, and 245 were treated with Combo.

### Synthetic Data Simulation

Data were simulated according to the model of the present embodiments as follows. The simulation process started by learning Mix on MSK-IMPACT panel data to obtain realistic estimates for the model's hyperparameters (10 clusters and 6 signatures using BIC) and parameters (cluster probabilities w, signature exposures π per cluster and the signatures themselves e). These estimates were used as a baseline for data simulation. In the simulations, the number of clusters L was varied from 5 to 9, by sampling clusters without replacement using the distribution w. The simulation process then assigned the clusters their corresponding weights from w, normalizing the sum to 1 w=(w₁, . . . , w_{L}). Let π=(π¹, . . . , π^{L}) denote the learned signature exposures over the selected clusters. Let

$${p}_{k} = {\sum }_{ℓ = 1}^{L} {w}_{ℓ} {π}_{k}^{ℓ} .$$

Next, the simulation process sampled without replacement K=4 signatures with probabilities p₁, . . . , p₆. The exposures were normalized per cluster over the selected signatures to sum to 1. This simulation setup was applied to generate 5,000 samples, similar to the number of samples in the MSK-IMPACT data. For each sample, the simulation process determined its number of mutations by sampling uniformly (with replacement) a sample from the MSKIMPACT data and adopting its number of mutations. The generative process of Mix is then used to sample mutations.

### Performance Evaluation in a Refitting Scenario

This Example describes application of the technique of the present embodiments to whole-genome and whole-exome data where information about active signatures exists. The evaluation procedures included generating sparse, down-sampled datasets to imitate the targeted sequencing data.

Down-sampling strategies. For evaluation purposes, targeted sequencing panels from higher coverage datasets were simulated. Two down-sampling strategies were used: (i) down-sampling WGS/WXS data by constraining the samples to target regions of MSK-IMPACT; and (ii) random sampling of an average of d mutations per patient. In detail, for each patient i nᵢ˜Pois(d) were sampled. Then nᵢ mutations were randomly sampled from the mutation set Oⁱ without replacement.

Reconstruction error. To compare methods in their ability to learn mutational signature exposures on sparse datasets, the reconstruction error (RE) obtained by each method was compared on a full dataset using relative exposures inferred on a down-sampled dataset. For ease of comparison, the signature matrix S was fixed to consist of known signatures from COSMIC. Since the full and down-sampled datasets have different numbers of mutations, they were compared only on their relative exposures. Let V be an N×M matrix where Vij is the number of times mutation category j is observed in tumor i in the full dataset, and let V be the normalized version of the matrix V such that each row sums to one. Given the N×K relative exposure matrix Ed computed on the down-sampled data, the reconstruction error was defined as

$$R E := {\vert \tilde{V} - {E}_{d} \cdot S \vert}_{1}$$

where |·|₁ is the L1 norm

Exposure Reconstruction error. Another reconstruction error measure used to compare signature learning from sparse data according to some embodiments of the present invention is exposure reconstruction error (ERE). Using the mutation matrix V and the signature matrix in the cancer type S, the non-relative (referred to as “true”) exposures E were learned using NNLS, which is a known method for learning exposures from rich data. Let E_{d} be the relative exposures computed on the down-sampled data, and let \tilde{E}_{d} be a normalized version of E, such that each row sums to one, the exposure reconstruction error is defined as:

$$E R E := {\vert \tilde{E} - {E}_{d} \vert}_{1} .$$

This measure is preferred in cases in which it is desired to know the exposures rather than the mutations, as the mutations can be noisy and it is less likely for mutational signatures to be able to reconstruct them with no error.

### Implementation Details

In this Example, Mix was implemented in Python 3. For NMF and KMeans the scikit-learn implementation was used. NNLS was taken from scipy [37]. The workflow is managed by Snakemake [38].

### Results

The method of the present embodiments is capable of elucidating the mutational signature landscape of input samples from their (sparse) targeted sequencing data. The method of the present embodiments was tested on synthetic data, down-sampled whole-genome and whole-exome data, and gene-panel data. The performances of the method of the present embodiments were compared to conventional methods. Mix was applied to learn parameters from synthetic data it generated. Mix was also used to reconstruct mutational signature exposures from down-sampled ICGC breast cancer [28] and TCGA ovarian cancer data [29], also applying it to another down-sampled data to cluster samples and predicting homologous recombination deficiency (HRD) status. Mix was also applied to the MSK-IMPACT Pan-Cancer targeted sequencing data [26, 27]. Its success was tested in discovering mutational signatures and in clustering patients. Mix was also tested in a clinical setting, aiming to predict the benefit of PARP inhibitor therapy for breast cancer patients and the benefit of immunotherapy for lung cancer patients.

### Mix Design

In the field of mutational signatures, known are NMF-based methods such as SigProfiler [4], and statistical analogs of NMF such as EMu [14] and signeR [16]. For these methods, the number of parameters grows linearly with the number of patients, as a consequence of learning an exposure vector for each patient. When using whole-genome or whole-exome data, each patient has many mutations, spanning most categories of mutations (usually 96 categories), allowing the accurate estimation of these exposures. In the increasingly available case of gene panel data, patients typically have less than 10 mutations, causing most categories to have zero counts, leading to a number of parameters that is larger than the number of data points. The SigMA algorithm [24], which was designed to predict HRD status in breast cancer samples, learns patient clusters on rich data from whole genome sequencing, then associates sparse samples with these clusters using a likelihood score, and finally applies a classifier to predict HRD status.

Unlike conventional techniques, Mix simultaneously learns signatures and soft clusters patients, learning exposures per cluster rather than per sample. Then, to obtain a unique exposure for each new patient, Mix soft-clusters the patient's mutations and takes a linear combination of all exposures according to their probability. With this, Mix also solves another problem of conventional methods, where adding a new patient requires learning a new exposure vector for it.

### Performance on Synthetic Data

The model of the present embodiments was applied to synthetic data created to have similar characteristics as the MSK-IMPACT data. Mix was evaluated in both estimating the number of clusters and signatures that underlie the data and learning the model's parameters. The results are summarized in Table 1.1, below, and show that Mix can accurately reconstruct the simulation parameters from sparse data. In 4 out of 5 settings, BIC was a good estimator for the hyperparameters, estimating the exact number of clusters and signatures. In 3 out of these 4 settings Mix perfectly reconstructed all clusters' exposures and signatures (average similarity 0:97) and in one of these settings Mix reconstructed 8 out of 9 clusters, and the remaining one was a duplicate. In one setting, Mix underestimated the hyperparameters, learning 5 clusters instead of 8 and 3 signatures out of 4. The missing signature, however, is involved in less than 5% of the mutations. Without this signature there are only 5 clusters with distinct exposures (similarity <0:95), supporting the inferred model.

TABLE 1.1

| Simulated | BIC inferred | Average | | Average | |
|---|---|---|---|---|---|
| hyper- | hyper- | cluster | # Unique | signature | # Unique |
| parameters | parameters | similarity | clusters | similarity | signatures |
| (5, 4) | (5, 4) | 0.97 | 5 | 0.99 | 4 |
| (5, 4) | (5, 4) | 0.97 | 5 | 0.99 | 4 |
| (6, 4) | (6, 4) | 0.99 | 6 | 0.99 | 4 |
| (7, 4) | (7, 4) | 1.00 | 7 | 1.00 | 4 |
| (8, 4) | (5, 3) | 1.00 | 5 | 0.99 | 3 |
| (9, 4) | (9, 4) | 1.00 | 8 | 1.00 | 4 |

Reconstructing Mutation and Exposure Profiles from Simulated Data

Mix was applied to simulated down-sampled data derived from whole-genome and whole-exome sequencing. In this application the full mutation profiles were available and were used to guide the evaluation. This Example focuses the experiments on exposure learning (refitting scenario). The signatures were fix to be the known active COSMIC signatures in the given cancer type.

Mix was trained using down-sampled data from 50% of the samples. Exposures were computed on down-sampled data for the remaining 50% of (test) samples. The average reconstruction error (RE) and exposure reconstruction error (ERE) are reported herein on the whole mutation catalog of the test samples. These experiments were repeated for average number of mutations per sample d ranging from 3 to 18, and the MSK-IMPACT region (panel) mutations, with an average of 5.6 and 4.5 mutations for WGS BRCA down-sampling and WXS OV down-sampling data, respectively.

The performance of Mix are compared against the widely used non-negative least squares (NNLS) approach. Given a mutation count matrix V and signatures H, NNLS extracts (non-negative) exposures W that minimize ∥V-WH∥₂. The comparison also included hard clustering inference scheme for Mix.

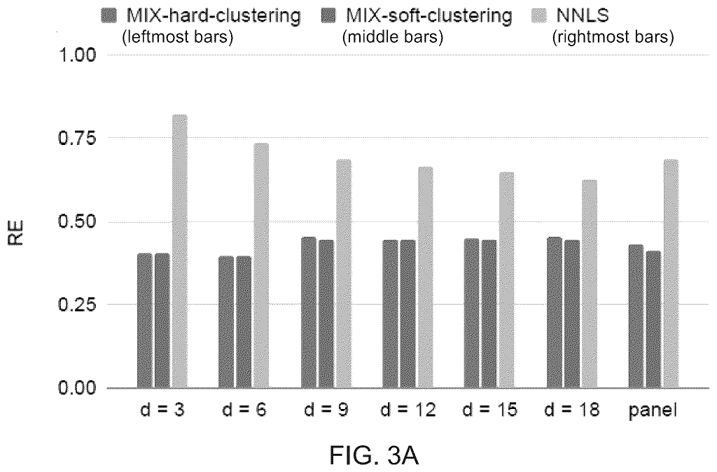

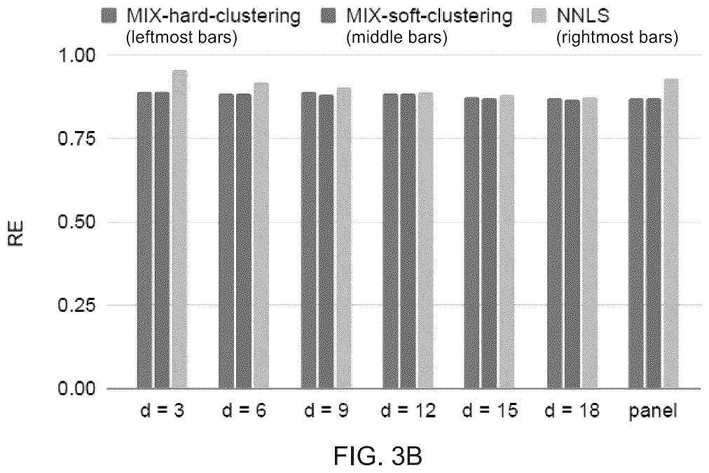

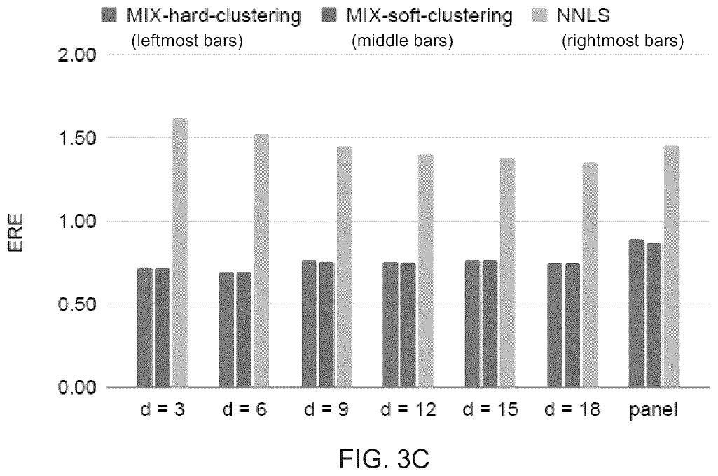

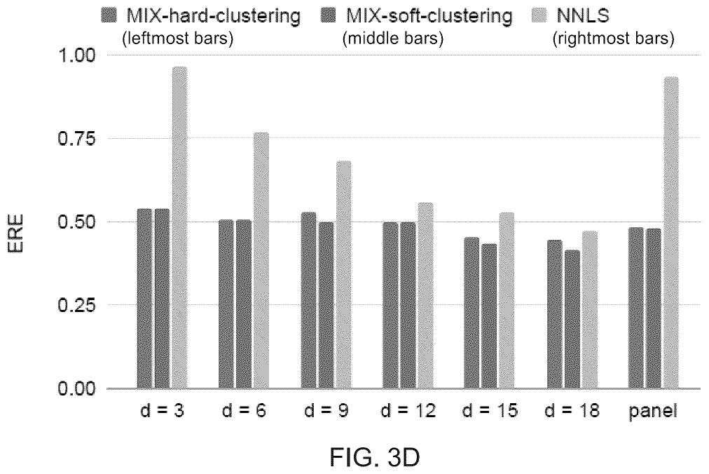

The results are shown in FIGS. 3A-D. Shown are RE and ERE for Mix (two variants) and NNLS across two datasets, breast cancer (FIGS. 3A and 3C) and ovarian cancer (FIGS. 3B and 3D), and seven of the down-sampling schemes. Out of the two Mix variants, the soft clustering inference of exposures displays better performance, and both outperform NNLS in all cases. Note that there is no decrease in reconstruction error when the number of mutations increases. Without wishing to be bound to any particular theory, it is assumed that is caused by noise in mutation data, which is mitigated when reducing the dimension of the data from mutation categories to signatures.

### Comparison to SigMA on Clinically-Relevant Data

Next, Mix was compared with and SigMA. To this end, Mix was trained using a panel down-sampling version of the BRCA data, with the 12 COSMIC signatures that are known to be active in this cancer type. In this application, BIC yields an estimate of 3 clusters which was used in the training of Mix. SigMA was trained on those 560 BRCA samples, along with 170 additional samples [24].

Both models were applied to panel down-sampling of 224WGS triple negative breast cancer samples, clustering them and predicting their HRD status. Signature 3 activity is known to be a good predictor of HRD [39], with 0.96 Area Under the ROC Curve (AUC) on this dataset when estimating its exposure using NNLS on the full (WGS) mutation data. For Mix, the status estimate was based on Signature 3 exposure using the soft clustering variant. For SigMA, the Signature 3 mva output was used. For completeness, an NNLS estimate of Signature 3 exposure on the panel down-sampling data was also evaluated.

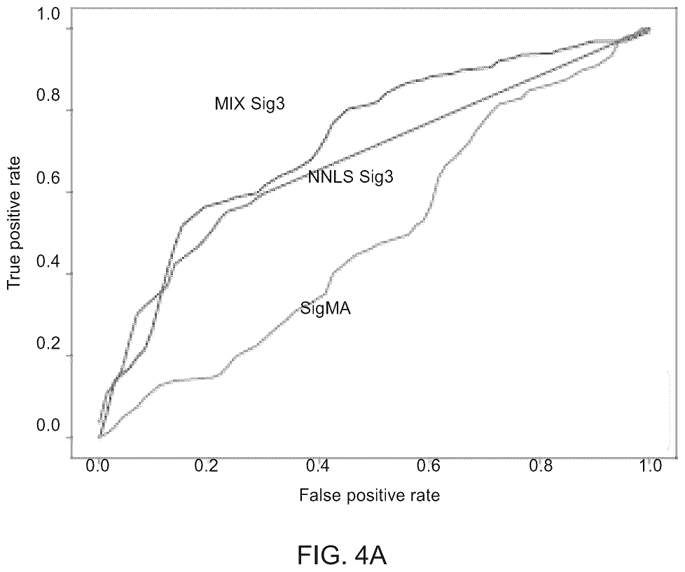

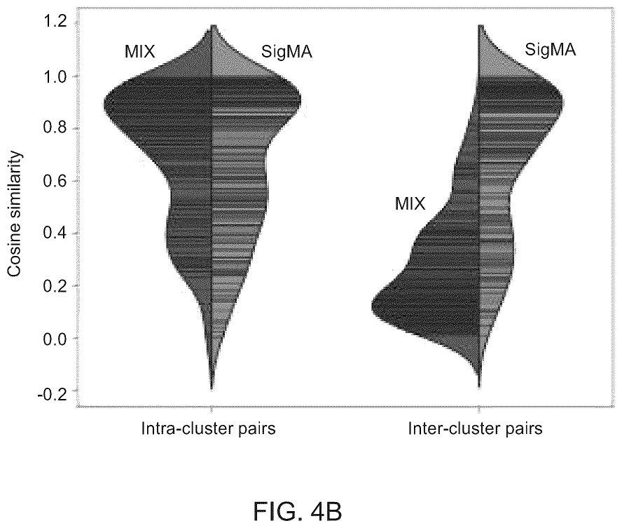

The results are shown in FIGS. 4A and 4B. FIG. 4A shows ROC curves for HR deficiency prediction based on Mix, SigMA and NNLS with AUCs of 0.73, 0.5 and 0.68, respectively, and FIG. 4B shows clustering quality of Mix and SigMA as measured by intra-cluster and inter-cluster cosine similarities.

The HRD status prediction ROC curves of the three methods are depicted in FIG. 4A with Mix showing a clear advantage over the two competing methods. When considering the performance of the three methods at low false positive rates (FPRs), 31% true positive rate (TPR) for Mix at 10% FPR was observed, which is on par with NNLS (34%) and higher than SigMA (12%); for an FPR of 20% the TPR of Mix increases to 58%, outperforming NNLS (48%) and SigMA (34%).

The clustering produced by the hard clustering variant of Mix was compared to the ‘categ’ output of SigMA. For each method, 200 intra-cluster sample pairs and 200 inter-cluster sample pairs were randomly drawn, and compared the distributions of similarities they induce. Specifically, the evaluation included computing cosine similarity between their exposures in the WGS data, obtained by NNLS with the 12 known COSMIC signatures in breast cancer. As shown in FIG. 4B, the intra-cluster pairs of Mix displayed substantially higher similarity than inter cluster pairs (0.69 vs. 0.27), while no such difference was observed for SigMA (0.65 vs. 0.66).

Learning Signatures and Patient Classes from MSK-IMPACT

Mix was applied to analyze 5931 samples from the MSKIMPACT dataset. Mix was trained with ten random initializations on number L of clusters ranging from 1 to 15 and number K of signatures ranging from 1 to 12 (up to 12 signatures are associated with these data according to COSMIC).

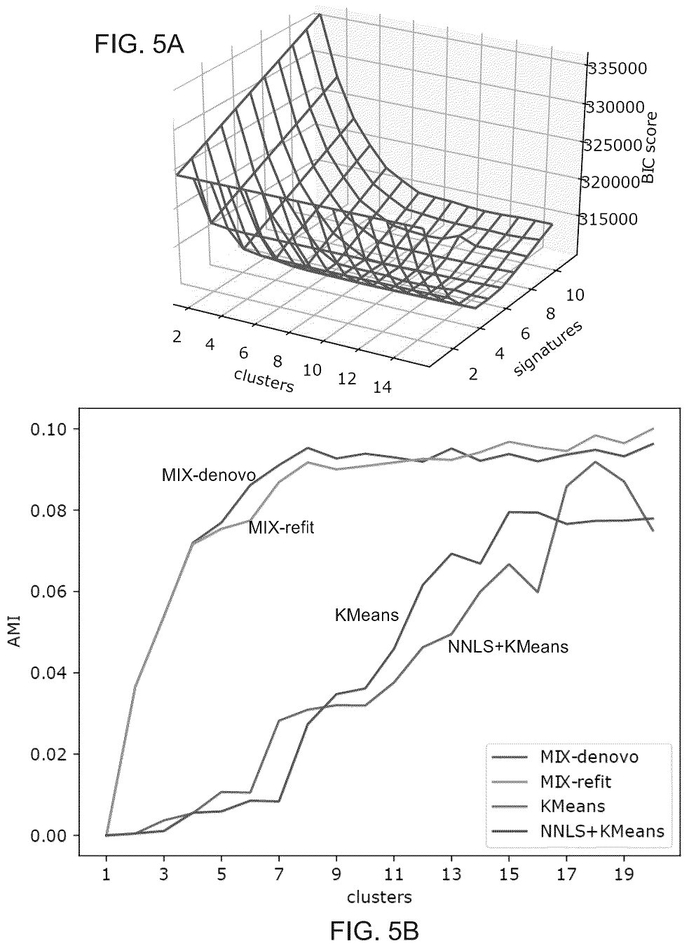

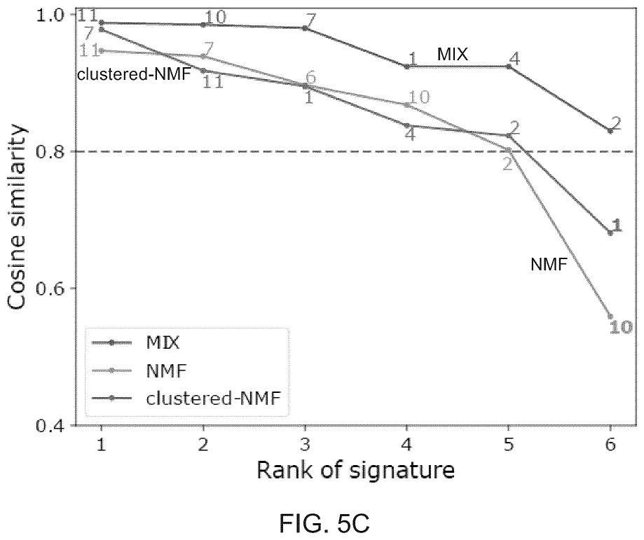

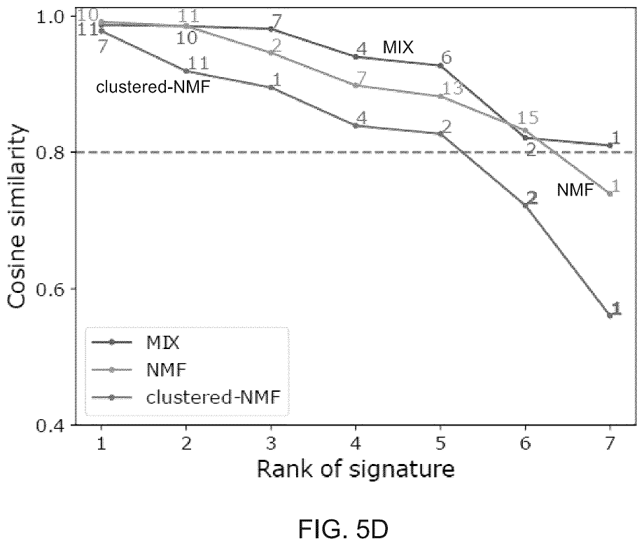

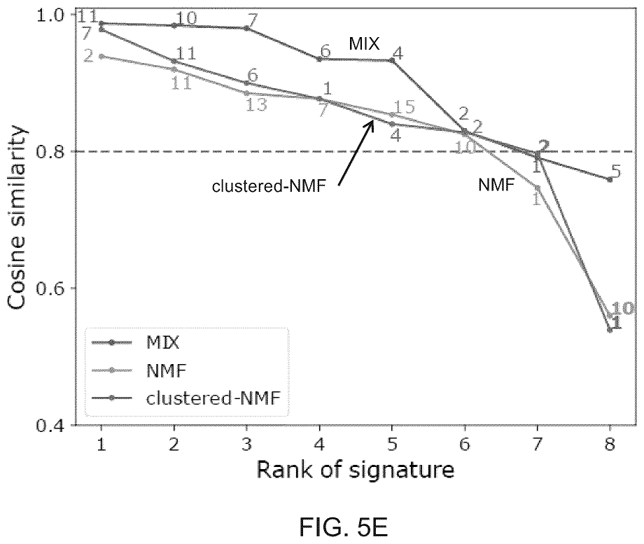

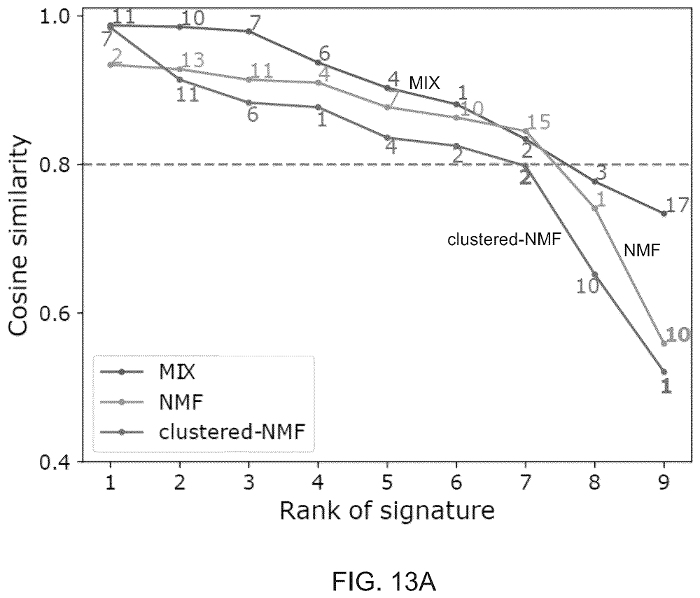

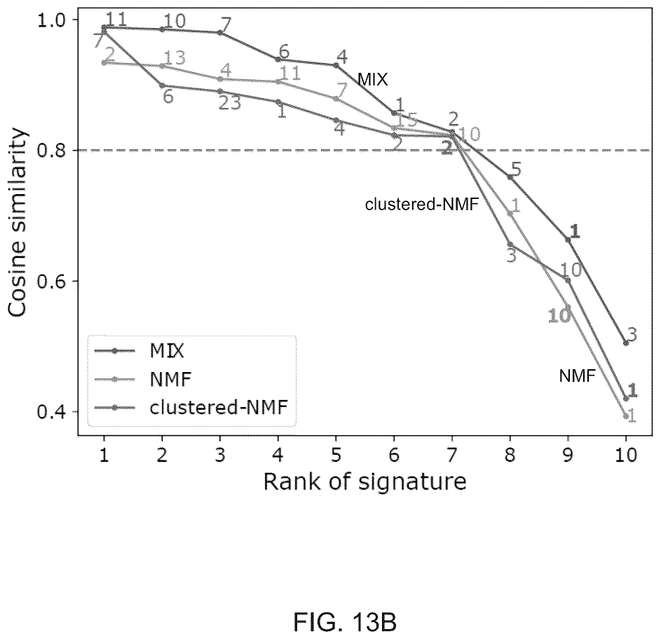

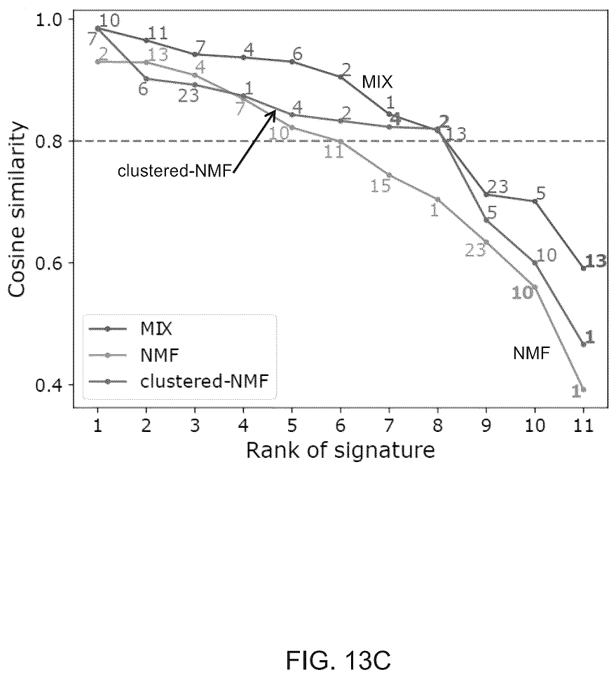

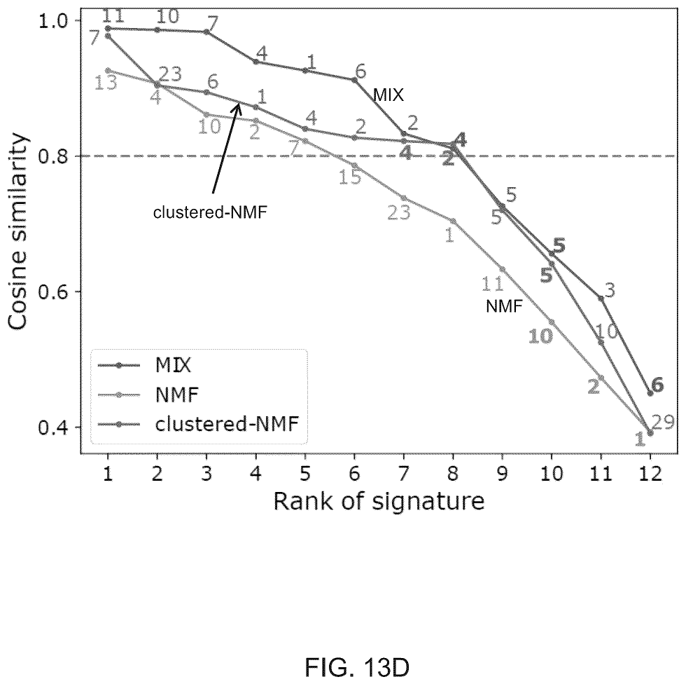

The performance evaluation on the MSK-IMPACT data is shown in FIGS. 5A-E and 13A-D. FIG. 5A shows hyper-parameter selection in Mix. Sown is a plot of BIC score (y-axis) as a function of the number of signatures (z-axis) and the number of clusters (x-axis). FIG. 5B shows AMI score as a function of the number of clusters for each model. FIGS. 5C-E show de-novo signature discovery from MSK-IMPACT panel data. Shown are sorted cosine similarities between learned signatures and most similar COSMIC signature (denoted next to each plot) for Mix, NMF and clustered NMF across a range of number of signatures (6-8 corresponding to FIGS. 5C, 5D, and 5E, respectively). FIGS. 13A-D show sorted cosine similarities as in FIGS. 5C-E except for signatures 9, 10, 11, and 12, respectively. Repeating signatures of the same model are in bold.

Using BIC L=10 and K=6 were found to be the optimal hyper parameters (FIG. 5A). A refitting version of Mix was also trained on this dataset with the known 17 COSMIC signatures and found L=7 using BIC.

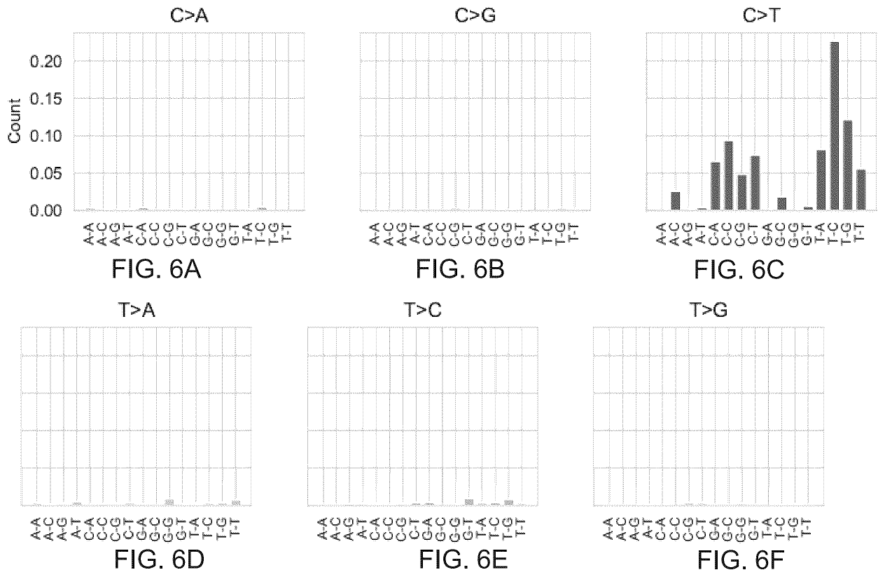

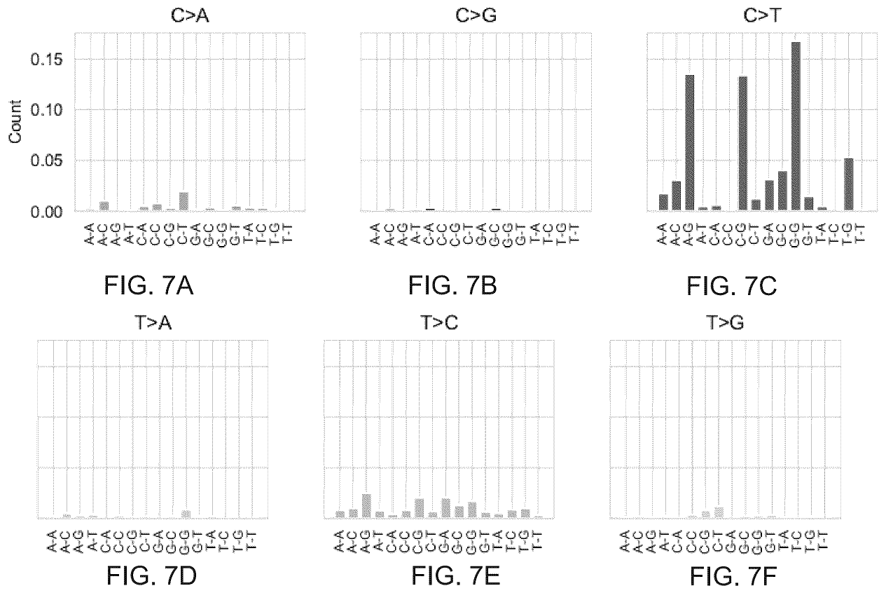

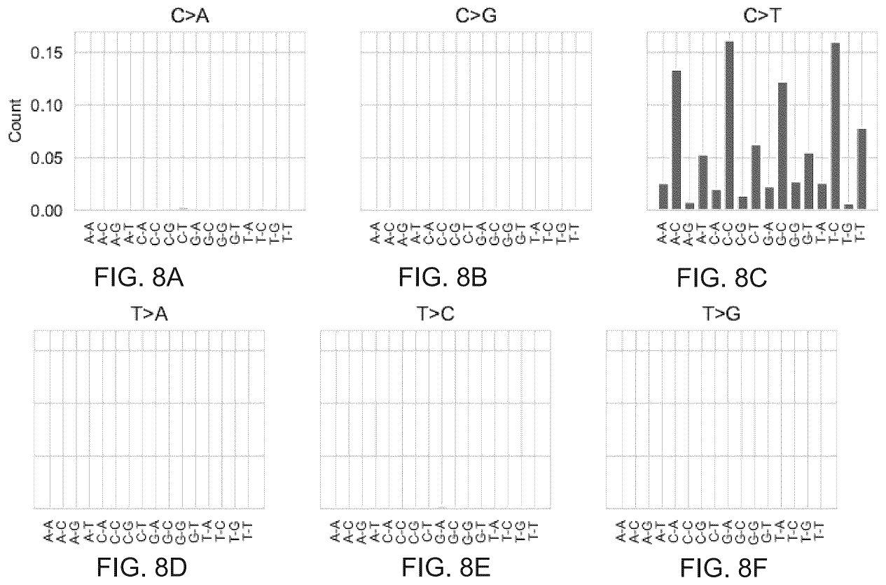

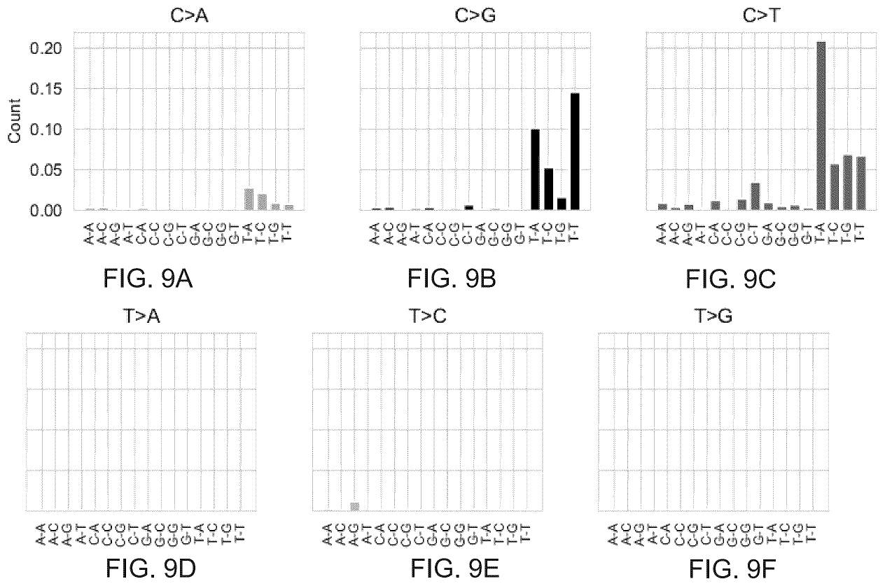

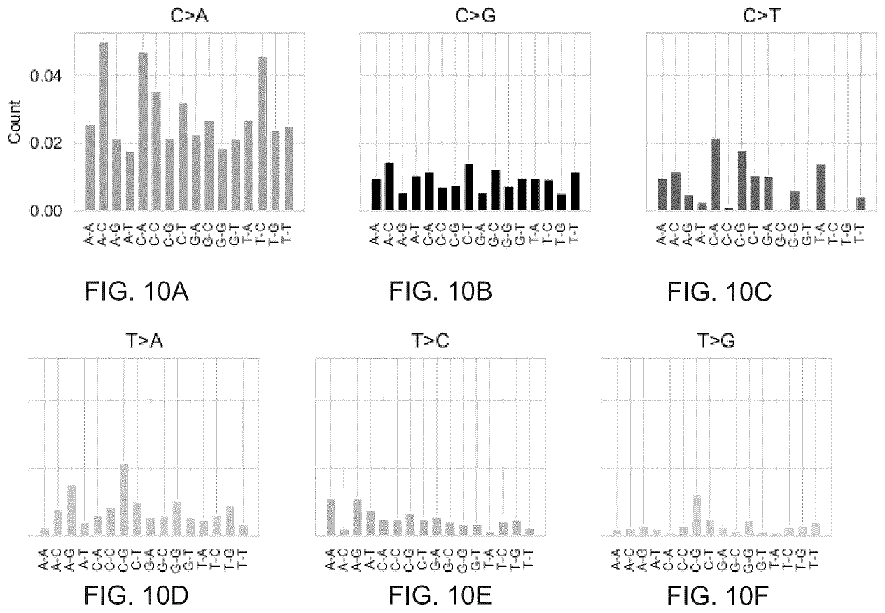

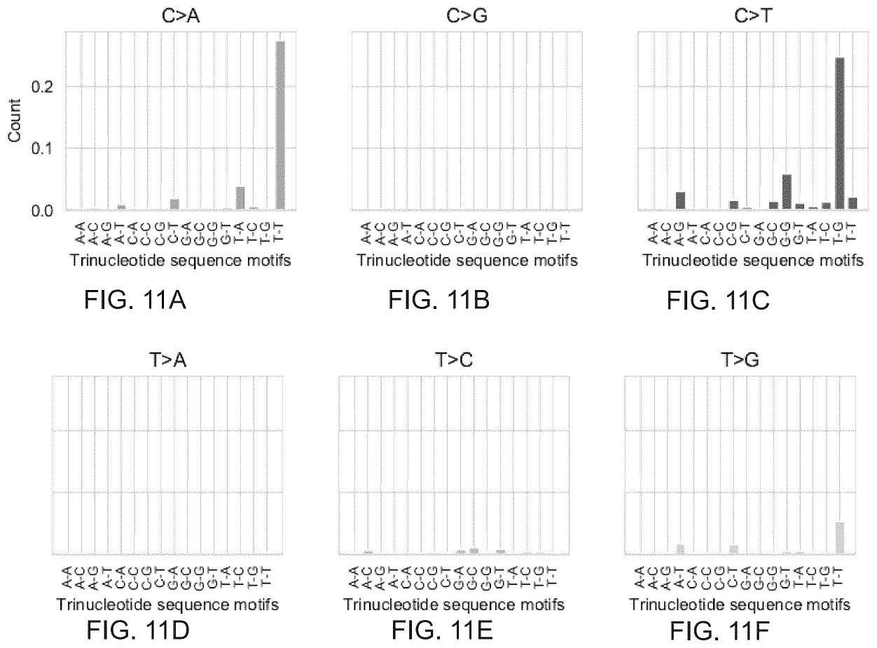

The six learned de-novo signatures are shown in FIGS. 6A-11F, where FIGS. 6A-F show a first signature, M1-Sig7 (0.98), FIGS. 7A-F show a second signature, M2-Sig1 (0.92), FIGS. 8A-F show a third signature, M3-Sig11 (0.99), FIGS. 9A-F show a fourth signature, M4-Sig2 (0.83), FIGS. 10A-F show a fifth signature, M5-Sig4 (0.92), and FIGS. 11A-F show a sixth signature, M6-Sig10 (0.99). Shown are distributions for the 6 de-novo signatures learned from MSK-IMPACT using Mix. For each signature M1-M6 the respective figures indicate the most similar COSMIC signature and the cosine similarity.

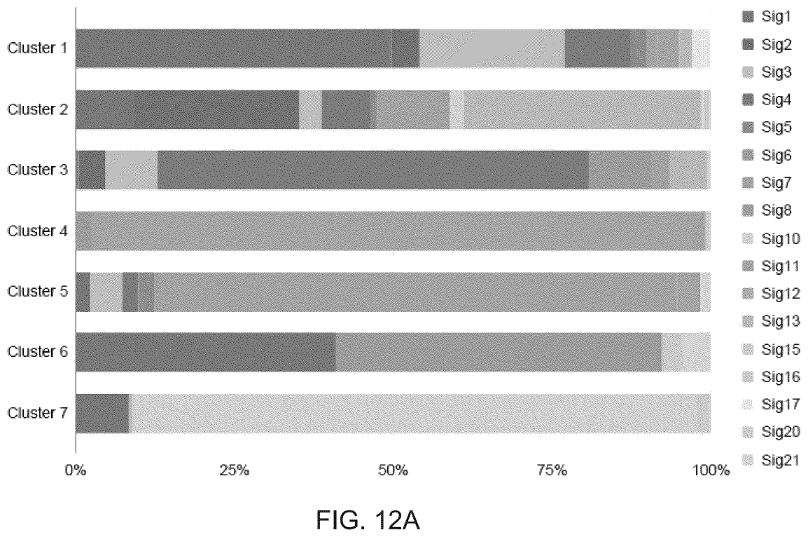

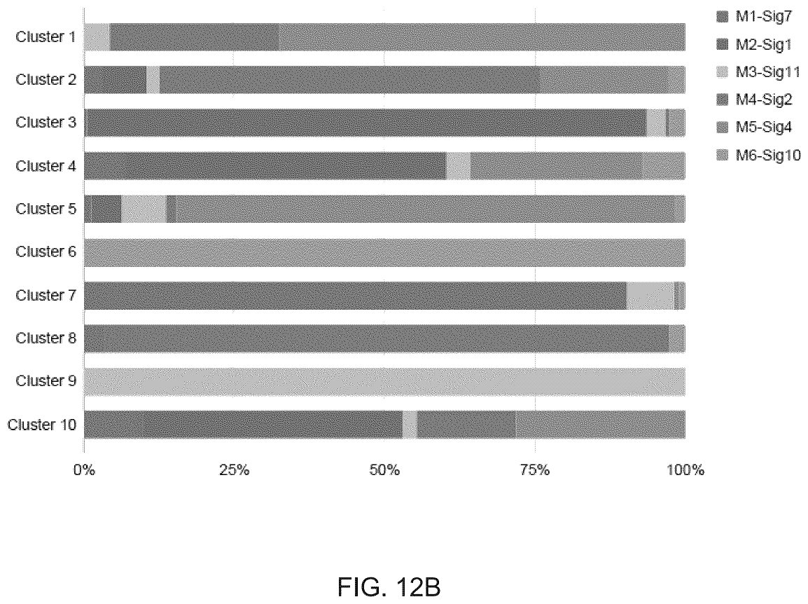

FIGS. 12A and 12B show signature distributions in the clusters Mix learned from the MSK-IMPACT data. FIG. 12A shows refitting clusters learned using the known 17 active COSMIC signatures, and FIG. 12B shows 10 clusters learned de-novo.

The BIC score is affected mostly by the number of signatures, with a minimum between about 5 and about 7, but less so by the number of clusters. The learned signatures were compared to the COSMIC signatures using the cosine similarity measure (FIGS. 5A-E and 13A-D). Mix accurately reconstructed 6-8 known signatures with cosine similarity above 0.8. The performance were compared to that of the standard NMF algorithm as well as to a clustered variant where meta-samples corresponding to each of the 18 cancer types was formed, and then the NMF was applied to these meta-samples. To form a meta-sample, all mutations of samples that belong to the corresponding cancer types were combined (in this Example, the samples' mutation counts were summed together). For these additional applications the number of signatures was varied from 6 to 8. For Mix the number of clusters in each application was optimized using BIC as described above.

Each algorithm was executed ten times with different (random) initializations and the run that yielded the best score (likelihood for Mix or approximation error for NMF) was chosen. Evidently, Mix dominates the conventional across the explored range, yielding a larger number of highly accurate and distinct signatures.

Mix was also compared to SigProfiler [4] and SigAnalyzer [40, 41], two variants of NMF that were designed specifically for the task of learning mutational signatures. Although they were not designed for sparse data, both tools were applied using their default settings. However, SigProfiler was too time consuming (expected running time of days to weeks to perform the experiments described in this Example), and SigAnalyzer gave inferior results to the NMF application reported in this Example (only two signatures, 1 and 7, consistently recovered with cosine similarity greater than 0.8).

Mix was also used to cluster samples, choosing for each sample the cluster with maximal posterior probability. The resulting clusters were scored against a benchmark clustering of the samples according to their cancer type with the adjusted mutual information (AMI) score (see, FIG. 5B). Predicting cancer type from targeted sequencing panels can be used for clinical purposes, as approximately 3% of tumors are of unknown primary origin and there has been a recent focus on developing methods to predict cancer type using mutations [43, 44].

The results were compared to those obtained by KMeans clustering of the original mutation count vectors as well as to a refined variant where NNLS was first applied to the data using the 17 active COSMIC signatures, and the resulting exposures were then clustered using KMeans. For Mix an additional refitting variant was presented. In this variant the signatures were set to be the 17 COSMIC signatures. This Example reports results with L=1-20 clusters, for de-novo Mix the number of signatures for each value of L was chosen using BIC. As the clustering of specific samples depends on their sparsity, also reported are AMI scores when focusing on samples with at least 10 mutations.

FIG. 5B demonstrates that the two Mix variants outperform the conventional methods in both settings. Note that the two Mix variants display similar performances, suggesting that Mix can cluster well even without prior knowledge. The fact that the Mix AMI scores converges for 6 clusters or more suggests that Mix is robust to the number of clusters being used.

### Predicting Immunotherapy Response of Lung Cancer Patients

The utility of Mix in additional clinical scenarios in which signature analysis is less abundant was also tested. Specifically, Mix was applied to 150 LUAD samples to predict durable clinical benefit to PD-L1 monotherapy treatment. The same de-novo was used and Mix models that were trained on the MSK-IMPACT pan cancer data in previous section were refitted. Most samples in the MSK-IMPACT data set are from LUAD patients (1277, 21%).

Tumor mutational burden (TMB) is one of the most widely known and analyzed genomic correlates with immunotherapy response. In addition, Rizvi et al. found that the exposure of Signature 4 was associated with response in non-small cell lung cancer. For the former, as targeted sequencing data were available, the plain mutation counts were used instead which were shown to be in high correlation with TMB [46]. For the latter, Mix was used to obtain signature exposures and compared to those derived using NNLS on the 17 COSMIC signatures active in the MSK-IMPACT dataset, or using SigMA.

The performance of each method using AUC were evaluated. Specifically, the AUC score of Signature 4 at predicting the treatment response is reported. For Mix in the de-novo setting, the signature which is most similar to signature 4, with cosine similarity 0.924 is reported. The AUC scores are 0.64, 0.63, 0.63, 0.6 for refit-Mix, de-novo-Mix, NNLS and TMB, respectively. The results suggest an advantage for Mix over alternative approaches in this setting.

### Discussion

One application of Mix is for predicting the benefit of drug treatments based on the inferred activities of relevant mutational signatures. In particular, this Example showed the ability of the model of the present embodiments to predict HRD status and hence the benefit of treatment with PARP inhibitors. The results were on a down-sampled whole-genome sequencing dataset where ground truth was determined by the HRDetect algorithm. HRDetect has shown promise at predicting response to PARP inhibitors and in stratifying triple-negative breast cancers by outcome.

Mix can also be trained on other HRD classifications or investigating discrepancies between Mix and HRDetect.

Mix can be applied on a dataset with sequencing data and PARP response. This Example also showed the ability of the model of the present embodiments for predicting the response to immunotherapy.

Beyond the prediction of signature exposures, Mix has the advantage of clustering the patients to potentially clinically-relevant groups. To showcase this relevance, this Example demonstrated a survival analysis of 1583 pan-cancer patients from whose mutation profiles are not used for the training process of Mix. Mix was applied in both the refitting and the de-novo settings, and assigned Mix cluster memberships to patients via hard clustering, in which each patient is assigned to the most likely cluster.

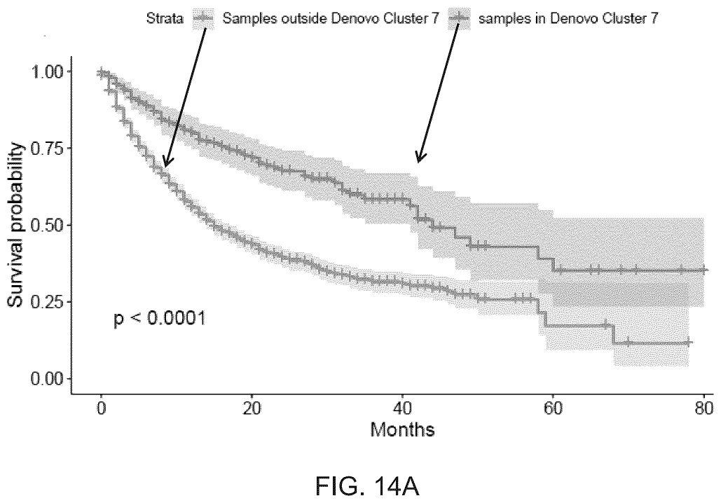

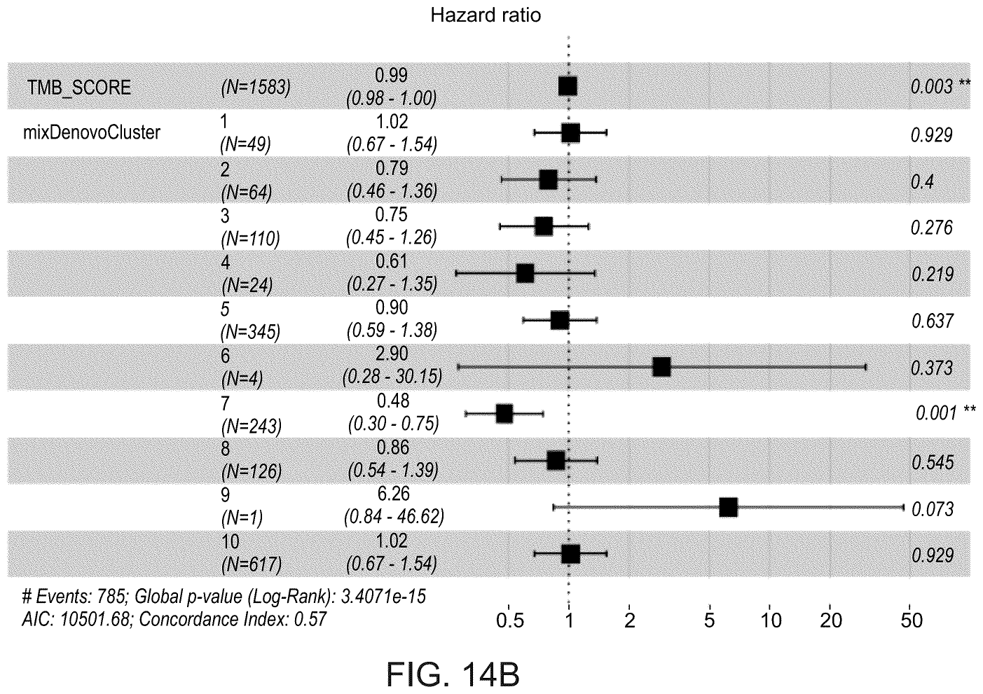

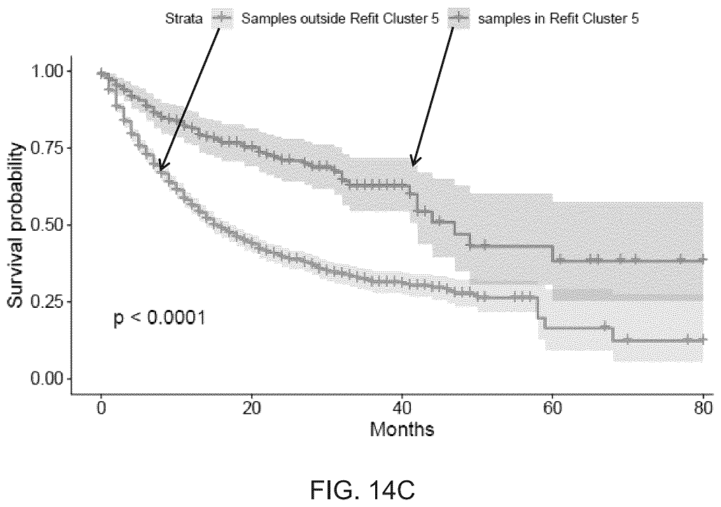

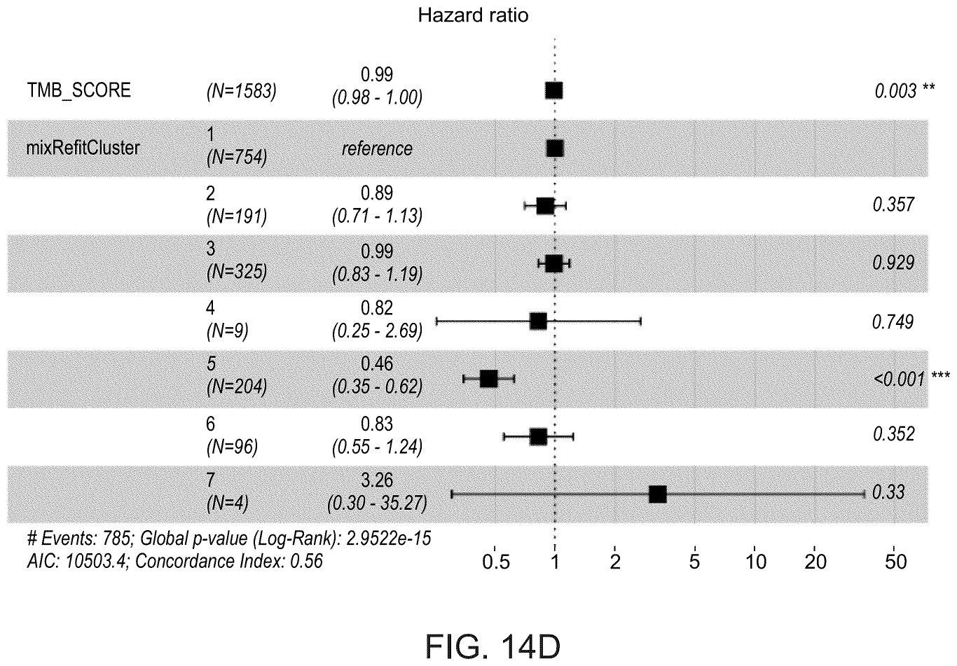

FIGS. 14A-D show Mix clusters which stratify pan-cancer patients into different survival groups. FIGS. 14A and 14C show Kaplan-Meier plot for de-novo Mix cluster 7 and refit Mix cluster 5 (p<0.0001, log-rank test), and FIGS. 14B and 14D show hazard ratios of TMB scores and Mix clusters (significance levels are computed using likelihood ratio tests).

Out of the seven refitting clusters, patients in cluster 5 have significantly better survival. The analysis indicates that the dominant signature in this cluster with exposure of 0.82 is Signature 7, which is associated with UVradiation. At the same time, 131/204 patients in the cluster are SKCM patients, which agrees with the previous finding that Signature 7 is correlated with better SKCM survival [47]. Out of ten de-novo clusters, patients in cluster 7 have significantly better survival. This cluster is again dominated by Signature 7 (exposure of 0.9) and 149/243 of its members are SKCM patients (FIGS. 14A-D). For comparison purposes, this Example evaluated other relevant clinical variables. It was observed that TMB scores do not stratify patients into statistically significant survival groups, nor age or gender.

It is preferred that the number of clusters be greater than the number of signatures. When it is desired to cluster samples, the number of clusters can be equal to the number of signatures, and the method can require a single signature with an exposure of 1 in each cluster.

### Conclusions

Sparse mutation data, as characteristic of targeted sequencing assays, is becoming increasingly available in the clinical setting with important applications in diagnosis and therapy. This Example presented a technique to model such data and derive the underlying mutational signatures, exposures and clinically-relevant predictions. The model of the present embodiments can directly capture sparse data without the need for pre-training on rich datasets. This Example demonstrated usage of the technique in a range of tasks, and also its favorable performance in comparison to existing methods.

This Example showed that the model of the present embodiments can predict HRD status in breast cancer, immunotherapy response in lung cancer, and patient stratification. In some embodiments of the present invention the analysis is supplemented by specific predictors for the tasks at hand, optionally and preferably using additional data (beyond signature exposure). The model of the present embodiments can optionally and preferably use of WGS/WXS data, when such are available, to improve the signature discovery.

### Example 2: Targeted Genomic Panels

This Example describes a method suitable for automatically designing targeted panels for the purpose of detecting a preselected mutational signature. This Example demonstrates that the method described herein can, in some cases, approach the performance of whole exome sequencing, which observes over ten times as much genomic material.

Over the past few decades, research has revealed that cancer is a disease characterized by the accumulation of mutations (Stratton et al., 2009; Garraway and Lander, 2013; Kandoth et al., 2013; Gerstung et al., 2020). Over the lifetime of an organism, cells acquire mutations at random positions in the genome. Certain mutations disrupt the function of cellular systems, and if certain cellular systems are disrupted simultaneously, a cell can lose its ability to regulate its rate of reproduction. Dysregulation of the reproductive cycle is one of a handful of “hallmark” qualities (Hanahan and Weinberg, 2011), which together transform a healthy cell into a cancer cell. These hallmark qualities are shared by all cancers, but they can be caused by random mutations in many respective genes. Furthermore, many such genes are mutated only at low frequency, implying that there are numerous combinations of mutated genes that can lead to cancer [e.g., see the analysis by Lawrence et al. (2014)]. The randomness involved in determining a cell's path to the disease state is responsible for part of the difficulty of treating cancer (Zugazagoitia et al., 2016).

Since cancer genomes vary so widely, the set of therapeutic targets does also, necessitating a diverse set of treatment strategies and engendering difficult decisions about which one to use for a given patient.

The random process by which mutations are acquired in the genome is not uniform. It is patterned (Helleday et al., 2014). For example, mutations acquired by smoking tend to produce a different pattern than those acquired from UV radiation (Nik-Zainal et al., 2015) or those acquired endogenously through errors made during genome replication (Tubbs and Nussenzweig, 2017), etc. Recent work demonstrates that identifying which of these mutational processes are active in a tumor genome provides a useful basis with which to categorize the diverse landscape of tumor phenotypes (Hoeck et al., 2019). Indeed, such activity can serve as an effective biomarker in the clinic. Past work has shown that certain mutational processes can indicate a tumor's vulnerability to particular therapies. One example is mismatch repair deficiency, which can indicate the effectiveness of checkpoint inhibitor immunotherapy (Le et al., 2017). A second is homologous recombination repair (HR) deficiency, which can indicate effectiveness of poly ADP ribose polymerase (PARP) inhibitor therapy (Farmer et al., 2005; Lord and Ashworth, 2017).

Designing and applying methods for the detection of mutational processes is known. One approach was pioneered in 2012 by Nik-Zainal et al. (2012), who utilized machine learning methods to extract the signature patterns of several mutational processes from aggregated tumor genome samples. Alexandrov et al. (2013b) formalize a mutational signature as a probability distribution over a set of mutation categories—that is, they suppose that a given mutational process can be identified by the frequency with which it causes each type of mutation. Their landmark article utilized distributions over 96 mutation categories, defined by all possible single base substitutions (SBS) with trinucleotide context. To infer the signatures from the data, they formulated a simple linear model of a tumor's mutations. In their model, a relatively small number of mutation signatures are shared across tumors, and the mutations of each tumor are given as a linear combination of these shared signatures plus some noise.

To realize this model, they apply non-negative matrix factorization (NMF) to genome-wide mutation counts on a large cohort of tumor samples. When applied to this problem, NMF infers a set of mutational signatures, as well as the activity of each signature on each genome in the cohort (often called the exposure). While other learning algorithms and schemes for categorizing mutations have been used, this approach remains the most common method of signature extraction in mutational signature analysis (MSA) studies (Alexandrov et al., 2013a, 2020; Kim et al., 2016). The current state of the art for SBS mutational signatures identified 49 signatures, extracted from an analysis of 23,829 tumors. Recent work has highlighted mutational signatures as a powerful diagnostic tool for clinical use, with several signatures implicated as potential therapeutic biomarkers (Davies et al., 2017; Van Hoeck et al., 2019). While these results are indicative of great promise for the future of cancer treatment, current clinical treatments are largely unable to take advantage of these advances. This is primarily due to the outsized sequencing requirements of the computational methods used in MSA studies (Alexandrov et al., 2013b, 2020) relative to current clinical sequencing practices (Frampton et al., 2013; Cheng et al., 2015). The “gold standard” data source for MSA studies is whole-genome sequencing (WGS). This is because WGS gives a complete and unbiased picture of the mutations present in a tumor genome, which affords increased accuracy during signature extraction. When WGS data are not available or in short supply, MSA studies also commonly use whole-exome sequencing (WES) as a data source. WES takes a subset of the genome that is smaller but still sizable—it is thus cheaper to sequence but still provides utility for signature extraction.

Conventional MSA studies use WGS or WES to obtain mutation counts in tumor genomes. WGS and WES are unavailable to most cancer patients-current clinical sequencing practices are commonly limited to targeted sequencing. This term refers to precisely-aimed therapeutic assays that sequence small pieces of the genome with known biological importance. This can provide valuable information about particular genes of interest, but provides a much more limited picture of the distribution of mutations on the genome which MSA seeks to assess. Targeted sequencing assays identify abut 1000 times fewer mutations compared to WGS (Nik-Zainal et al., 2020) and about 100 times fewer mutations than WES. Furthermore, while there have been calls to make large-scale sequencing assays clinically available, ramping up production of sequencing data in the clinic will take time and resources. Despite the decreasing costs of sequencing itself, providing large-scale genomic sequencing to patients requires infrastructure for analysis, interpretation, and storage of the resulting data. Thus, WGS in particular is unlikely to be offered as a routine clinical diagnostic for many years to come (Nik-Zainal et al., 2020). In the meantime, the therapeutic impact of MSA in the clinic is tied to the important open problem of inferring signature activity from targeted sequencing assays.

Recent studies have designed methods to detect mutational signature activity from clinically accessible targeted sequencing assays. Campbell et al. (2017) found that in patients with hypermutation, panel data could be used to infer exposures using standard methods (Rosenthal et al., 2016). Gulhan et al. (2019) introduced SigMA to detect signature activity indicative of homologous recombination repair deficiency, with a specific focus and application to gene panel data. Sason et al. (2020) introduced Mix to infer mutational signatures and their exposures in clusters of patients for use on datasets with few mutations per patient. In general, these studies make methodological modifications to account for the limited sample of the genome afforded by targeted sequencing assays. Even still, these methods are constrained by which regions of the genome are sampled. In particular, these works have sought to detect signature activity from two existing targeted sequencing assays: the Memorial Sloan Kettering Integrated Mutation Profiling of Actionable Cancer Targets (MSK-IMPACT) (Cheng et al., 2015) and FoundationOne Panel Sequencing (Frampton et al., 2013). These panels are designed to identify specific actionable mutations, which are most commonly found in genes linked with cancer. This goal is fundamentally different from the goal of MSA, which seeks to analyze the distribution of mutations over the genome. Accordingly, such panels likely provide a biased view of the true mutation distribution. This is evidenced by a recent prominent study which showed that mutations in cancer genes (which are common locations of actionable mutations) are subject to powerful selective pressure due to their impact on the fitness of a tumor. The authors showed that this can result in distributions of mutations that are not representative of the underlying mutational signatures (Temko et al., 2018). Thus it is likely that other regions of the genome are more suitable for interrogating the genomewide distribution of mutations.

One study contained a short analysis to identify a panel-sized set of genes, which could be used to detect one specific mutational signature (Polak et al., 2017). This analysis did not consider noncoding regions as candidates for the panel. Perner et al. (2020) recently introduced mutREAD, an assay for detecting mutational signature activity, but their approach concerns the method by which the DNA is sequenced rather than panel construction.

This Example presents a machine learning procedure referred to as ScalpelSig (Scalar projection Panels for mutational Signatures). The procedure learns from data to design genomic panels optimized for the detection of activity from an input mutational signature.

The term “genomic panel” is used to refer to a set of genome regions (including both coding and noncoding regions, in contrast to a gene panel) whose sequence can be used as a biomarker. The machine learning procedure was constructed with a goal of increasing clinical access to MSA, and as such all panels considered in the present Example are small enough to be sequenced and analyzed using current clinical infrastructure.

The machine learning procedure of the present embodiments considers the whole genome (and not just coding regions), and considers arbitrary mutational signatures rather than just those of homologous recombination repair.

The machine learning procedure of this Example was trained on a large cohort of breast cancer whole genome sequences and evaluate its performance on held-out data. Performance is evaluated below by extracting signature exposures from the discovered panel regions with standard methods and comparing the signature activity within panel regions to the ground truth of genome-wide signature activity. This performance is compared against two benchmarks: the MSK-IMPACT panel and a randomized baseline. Panels designed by ScalpelSig afford superior accuracy for signature detection in five out of six examined signatures. We additionally analyze the generalizability and robustness of our algorithm. This Example demonstrates that ScalpelSig's increased accuracy over baselines is maintained on an independent breast cancer dataset and for a wide variety of parameterizations.

### Methods

A mutational signature (or signature for case of exposition) is defined as is seminally defined by Nik-Zainal et al. (2012) and Alexandrov et al. (2013a,b). A signature is a multinomial distribution over a set of mutation categories. The most commonly studied mutation categories are SBS and their immediate 50 and 30 flanking bases (Alexandrov et al., 2013b), leading to 96 categories arising from six canonical SBS categories C>G, C>A, C>T, T>A, T>C, and T>G, and four possible 50 and 30 flanking bases. For example, the A [C>T] G mutational category refers to a C>T SBS immediately flanked by an A (50) and a G (30).

Each tumor or sample (belonging to a patient), whose genome is sequenced, can be summarily described by a 96-dimensional count vector of observed SBS belonging to each mutational category. MSA assumes that each observed mutation is emitted by a unique mutational signature, and it is generally assumed that only a few signatures are active in a tumor and in a cancer type. Briefly, MSA usually involves (1) deriving the distributions of the latent mutational signatures active in a cohort of samples; and/or, (2) determining the exposure each sample has to each signature—the proportion of mutations in each sample emitted by each signature.

This Example considers problems relating to the latter, more clinically relevant problem where a candidate set of potentially active mutational signatures in each sample is given and known. We perform analysis with respect to a canonical and widely adopted set of signatures determined by the Catalogue of Somatic Mutations in Cancer (COSMIC) ver. 2, and will always refer to (and index) COSMIC signatures by their numerical names (e.g., Signature 1) (Tate et al., 2019).

A panel optimized for detecting signature activity preferably consists of a small set of genomic regions which, when sequenced, allow the accurate identification of genome-wide patterns of mutations.

Jiao et al., 2020 showed a relationship between cancer type and regional mutation density. Other regionally varying chromatin features are discussed in Polak et al., 2015. Many mutational signatures are tied to molecular mechanisms which vary in their activity over the genome (Haradhvala et al., 2016; Morganella et al., 2016). These findings suggest that some mutational processes may prefer to cause mutations in specific regions of the genome. Therefore, some regions of the genome may be far more informative than others for assaying the activity of these processes and their associated signatures.

These informative regions poses a significant challenge, in part, due to the technical attributes of NMF, the most widely-used algorithm for signature extraction (Alexandrov et al., 2013a; Kim et al., 2016). NMF depends only on genome-wide mutation counts as input. Thus, NMF determines only the counts of mutations attributed to each signature and is agnostic to the location of mutations in the genome. This presents a barrier for understanding the regional distribution of signature activity. NMF is computationally intensive, and its resource requirements are amplified within the present use case. Broadly, the outputs of NMF lack robust structure: solutions are nonunique, the loss surface is nonconvex, and several random initializations of the algorithm or specialized initialization approaches (Boutsidis and Gallopoulos, 2008) are required to obtain a reliable result. Thus, it is computationally intractable to utilize NMF to compute signature activity and explore the combinatorial space of possible panels.

Instead of finding regions that assay the activity of all signatures at once, the method of the present embodiments breaks the problem down into distinct binary classification tasks. In clinical applications, signature activity serves primarily as a biomarker to decide whether a patient should receive a particular tailored treatment. In this use case, it is desired to have a panel that detects whether one specific signature has substantial activity on a given genome, rather than an estimate of the absolute number of mutations (or exposure) attributed to a collection of signatures.

Given a mutational signature, ScalpelSig designs a genomic panel optimized to distinguish samples where said signature is substantially active from those where it is inactive. ScalpelSig designs such a panel by selecting a set of discerning windows over the genome. In some embodiments of the present ScalpelSig includes the following operations: (i) divide the genome into nonoverlapping windows of a fixed size, (ii) compute, with a window scoring function, a measure of mutational signature activity in each window across a given cohort of samples, and (iii) combine the highest scoring windows to generate and output a genomic panel of a given fixed size.

### Scalar Projection Panels for Mutational Signatures

To efficiently optimize over the combinatorial space of possible panels, ScalpelSig uses scalar projection as a heuristic measure of mutational signature activity. Unlike conventional techniques for detecting signature activity, scalar projection has a closed form algebraic expression and can be computed deterministically in constant time. It also has robust structural guarantees. In particular, it is a linear transformation. These properties make optimization tractable, and this Example shows that scalar projection is indeed a suitable measure of signature activity. For vectors u and v, we write the scalar projection of u onto v as:

$${proj}_{v} ( u ) = \frac{1}{‖ v ‖} 〈 v , u 〉 .$$

The scalar projection of mutation category counts onto a mutational signature gives the magnitude of the least-loss signature activity vector, which in the present Example is the vector that results from scaling a mutation signature by its exposure, if the signature was the only active signature in the sample. We formalize this result in a Lemma, below.

Lemma. Given any mutation count vector c and a signature vector q, the magnitude of the least loss exposure vector in the direction of q is given by the scalar projection of c onto q, written as

$${proj}_{q} ( c ) = \frac{1}{‖ q ‖} 〈 q , c 〉 .$$

Proof. We seek the exposure a such that the residual between c and aq (the exposure vector) is minimized. Writing ∥·∥ to mean the 2-norm, we wish to solve minₐ ∥c-aq∥, or equivalently:

$${min}_{a} {‖ c - aq ‖}^{2} . \qquad ( EQ . 2.1 )$$

Writing EQ. (2.1) as an inner product, yields:

$${‖ c - aq ‖}^{2} = 〈 c - aq , c - aq 〉 = {a}^{2} {‖ q ‖}^{2} - 2 a 〈 q , c 〉 + {‖ c ‖}^{2} . \qquad ( EQ . 2.2 )$$

EQ. (2.2) is quadratic in a. Thus, we solve for a by taking the derivative with respect to a and setting it to be 0, which gives 0=2a∥q∥²−2 [symbol] q, c [symbol] . Rearranging, we find the solution, a= [symbol] q, c [symbol] /∥q∥². Notice that a is indeed the least-loss exposure and is non-negative since q and c are non-negative.

The exposure vector aq is given by the projection of c onto q. Writing the unit vector in the direction of q as:

$$\hat{q} = \frac{q}{‖ q ‖} ,$$

we set

$$aq = \frac{〈 q , c 〉}{{‖ q ‖}^{2}} q = \frac{〈 q , c 〉}{‖ q ‖} \frac{q}{‖ q ‖} = {proj}_{q} ( c ) \hat{q} . \qquad ( EQ . 3 )$$

Thus, the magnitude, proj_{q}(c), of the least-loss exposure vector, aq, relates to the value of the least-loss exposure, a, by a constant factor, 1/∥q∥, with a=(1/∥q∥)proj_{q}(c). This proves the Lemma.

Given a mutational signature q, a training set S of samples with subset A that has signature q active, and a maximum panel size N, ScalpelSig designs and outputs a panel, P, that distinguishes samples with substantial signature activity from those without. ScalpelSig designs such a panel by selecting an optimally-scoring subset from the set of all nonoverlapping genome windows of a given fixed width.

Let a mutational signature q be a vector representing a multinomial distribution over the standard 96 mutation categories described in Alexandrov et al. (2013b), and let cⁱ_{w} represent the 96-dimensional vector of mutation category counts that fall within genome window w for patient i. We define a panel P as a set of genome windows. Then, we denote the mutation category counts for patient i over panel P, to be

$${c}_{P}^{i} = {\sum }_{w ∈ P} {c}_{w .}^{i}$$

Using scalar projection as a heuristic for signature activity, ScalpelSig seeks to find a panel where the estimated activity of q is high only in samples where q is known to have substantial activity on the samples in A (e.g., whole genome). Formally, ScalpelSig solves the following optimization problem to find a genomic panel P that best detects the activity of signature q:

$$\begin{matrix}{maximize}_{P} & {\sum }_{i ∈ A} {proj}_{q} ( {c}_{P}^{i} ) − {\sum }_{j ∈ S ∖ A} {proj}_{q} ( {c}_{P}^{i} ) , \\ subject to & \vert P \vert = N .\end{matrix} \qquad ( EQ . 2.4 )$$

By the linearity of scalar projection, we have, for each sample i,

$${proj}_{q} ( {c}_{P}^{i} ) = {proj}_{q} ( {\sum }_{w ∈ P} {c}_{w}^{i} ) = {\sum }_{w ∈ P} {proj}_{q} ( {c}_{w}^{i} ) . \qquad ( EQ . 2.5 )$$

Thus, the objective, EQ. (2.4), can be rewritten as:

$$\begin{matrix}{maximize}_{P} & {\sum }_{w ∈ P} \{ {\sum }_{i ∈ A} {proj}_{q} ( {c}_{w}^{i} ) − {\sum }_{j ∈ S ∖ A} {proj}_{q} ( {c}_{w}^{i} ) \} , \\ subject to & \vert P \vert = N .\end{matrix} \qquad ( EQ . 2.6 )$$

This formulation of the problem allows optimization without exploring the large combinatorial space of possible panels. Instead, ScalpelSig determines an optimal panel by simply selecting the top scoring N windows for the window scoring function,

$$h ( w ) = {\sum }_{i ∈ A} {proj}_{q} ( {c}_{w}^{i} ) - {\sum }_{j ∈ S ∖ A} {proj}_{q} ( {c}_{w}^{i} ) . \qquad ( EQ . 2.7 )$$

The contrastive definition of this window scoring function naturally mitigates the impact of background noise, that is, enrichment of signature-associated mutation types for spurious reasons such as underlying nucleotide composition. To see this, observe that if a window contains spurious enrichment of a set of mutation types, one would expect inactive samples to have about as many mutations of these types as active samples. As a result, the second term in the equation (the sum of scores from inactive samples) is expected to be high, thus discouraging the selection of such a window.

Given that tumors have widely varying mutation rates, the method of the present embodiments optionally and preferably ensures that the panel is not tailored to patients and windows with the highest numbers of total mutations. To this end, the Inventors introduce a parameter α∈(0, 1] and re-parameterize the window scoring function to be:

$${h}_{α} ( w ) = {\sum }_{i ∈ A} {{proj}_{q} ( {c}_{w}^{i} )}^{α} - {\sum }_{j ∈ S ∖ A} {{proj}_{q} ( {c}_{w}^{i} )}^{α} . \qquad ( EQ . 2.8 )$$

Setting α=1 is equivalent to EQ. (2.7), and setting a=0.5 applies square roots to each of the summed terms. With α=0.5, the scoring function down-weighs the contribution from samples that have anomalously high projections. This yields a window scoring function that favors windows with high activity in most active samples and not windows with high activity in only a few samples in the training set. The present embodiments contemplate any value for the a parameter. Below, the values α=1 and a=0.5 are considered as representative examples.

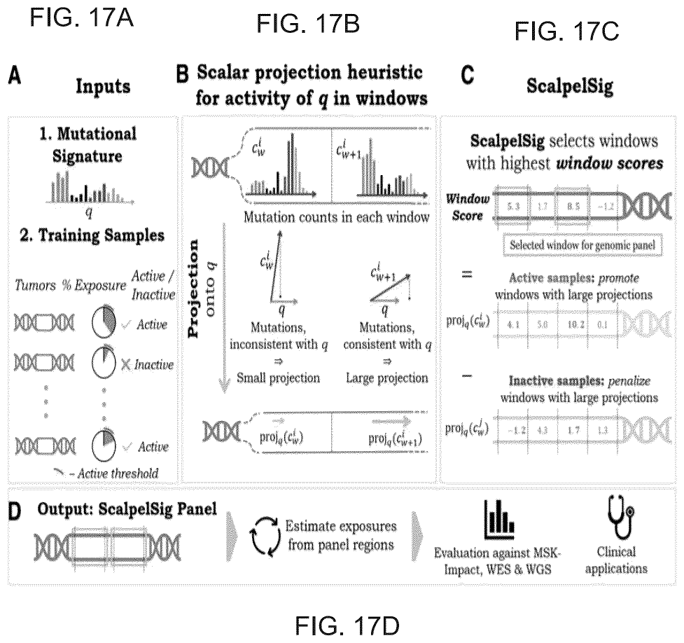

The above method is schematically illustrated in FIGS. 17A-D. The method employs a machine learning procedure. FIG. 17A illustrates the input to the machine learning procedure, including the mutational signature q and the set S of training samples with WGS data. Signature exposures are estimated for training samples. Samples which have more than 5% of mutations attributed to signature q are labeled active and inactive otherwise. FIG. 17B illustrates an operation in which each genome window's mutation count vector is projected onto q to provide a measure of signature exposure. The value of the scalar projection is highest when the given window has a high number of mutations with a distribution of mutation categories similar to q. FIG. 17C illustrates an operation in which the machine learning procedure evaluates windows with a contrastive window scoring function [see EQ. (2.8)] that encourages the selection of windows with large projections in active samples and small projections in inactive samples. Given a panel size parameter N, machine learning procedure combines the N top scoring windows into a panel. FIG. 17D illustrates the obtained panels which are suitable for detecting signature activity with improved accuracy and require considerably less genomic material than WES or WGS. Thus, the method of the present embodiments offers an improved, clinically accessible assay of signature activity.

### Mutational Signatures and Breast Cancer Cohorts

This Examples primarily presents analysis of a publicly available cohort of 560 breast cancer genomes (Nik-Zainal et al., 2016). The Inventors selected this dataset because of its large number of samples, and because many different mutational signatures are typically active in breast cancer (Tate et al., 2019), allowing for a varying set of test cases. The motivation of using mutational signatures as clinical biomarkers is particularly relevant to breast cancer, in which HR deficiency (and its associated mutational signatures) is a promising biomarker for PARP inhibitor therapy (Davies et al., 2017). WGS was performed on each sample. The dataset contains about 3.5 million total mutations categorized into the standard 96 categories used in Alexandrov et al. (2013b).

To further assess the generalizability of ScalpelSig, the Inventors also evaluated genomic panels learned from the above cohort (Nik-Zainal et al., 2016) on a completely held-out cohort of 237 WGS samples from Staaf et al. (2019).

### Computing Mutational Signature Exposures

For the estimation of mutational signature exposures on whole genomes, as well as all ScalpelSig panels and other baselines, the Inventors developed an open-source Python implementation of the SignatureEstimation framework (Huang et al.). For each sample in each cohort, the Inventors computed exposures to mutational signatures taken from COSMIC (ver. 2), a curated mutational signature repository (Tate et al., 2019). This Example refers to (and indexes) COSMIC signatures by their numerical names (e.g., Signature 1). Only 13 of the 30 COSMIC signatures are known to be active in breast cancer: Signatures 1, 2, 3, 5, 6, 8, 10, 13, 17, 18, 20, 26, 30 (Tate et al., 2019). Accordingly, from each sample's genome-wide mutation counts, the Inventors computed exposures solely to these signatures. From these exposures, the Inventors defines a sample as active for a signature if the exposure of that signature is responsible for 5% or more of the total mutations (compared to all other signatures), and inactive otherwise.

### Evaluation of Panels

For each signature of interest, the Inventors evaluated a ScalpelSig panel by how well it detects mutation signature activity in held-out samples. A primary measure of performance is defined as follows: given a sample, can one determine whether a signature is active on the whole genome by looking solely at panel regions obtained from ScalpelSig. This question is formalized with the following classification task: for each sample in the test set predict whether a signature of interest is active on the whole genome, given the signature exposure estimated from mutations that fall within a ScalpelSig panel.

### Comparison of ScalpelSig Against MSK-IMPACT and Other Baselines

The Inventors primarily analyzed the performance of ScalpelSig on the cohort of 560 samples from Nik-Zainal et al. (2016). The experiments use stratified sampling to split the data into training and testing sets. In each experiment the mean performance across 15 random test/train splits is reported. For each signature and unless otherwise noted, ScalpelSig uses 90% of samples as a training set to design a panel. Afterward, signature exposures are extracted from panel regions on the remaining samples. The Inventors measured how well these panel exposures distinguish active from inactive samples by computing area under the precision-recall curve (AUPR).

To further demonstrate the effectiveness of ScalpelSig panels, the Inventors also used Spearman's rank correlation to measure the strength of the relationship between exposures computed solely from mutations that fall within panel regions and exposures computed from whole genome mutation counts. This metric is independent of the formalism of active and inactive samples. The Inventors additionally tested multiple values for the threshold at which samples were considered active. The primary set of experiments were performed on 2.5 Mb ScalpelSig panels. This size was chosen to match the size of the MSK-IMPACT panel (Cheng et al., 2015). ScalpelSig was evaluated with α∈{0.5, 1} and the window size was set to 10 Kb for all experiments.

The Inventors performed experiments only for mutational signatures that are active in >5% of samples in the breast cancer cohort (Alexandrov et al., 2013a). Signatures 1 and 5 were omitted since these signatures are known to be endogenous and “clock-like” and are expected to be present in all samples (Alexandrov et al., 2015).

Thus, experiments were performed to evaluate ScalpelSig panels optimized for the detection of each of the six remaining signatures (2, 3, 8, 13, 18, and 30). The number of active samples for each signature is provided in Table 2.1, below. Specifically. Table 2.1 lists the number of active samples in the 560 breast cancer samples for the mutational signatures known to be present in breast cancer. A signature is active if that signature is responsible for 5% or more of the total mutations in the sample. Signatures 6, 10, 17, 20, and 26 are active in fewer than 5% of samples and, thus, are not considered in this study. Signatures 1 and 5 are also not considered because they are expected to be active in every sample (Alexandrov et al., 2015).

The Inventors compared ScalpelSig panels against three benchmarks, each an alternative approach that sequences fewer bases than WGS: the MSK-IMPACT panel (Cheng et al., 2015), whole exome sequencing (WES), and a randomized baseline. To compare against the MSK-IMPACT panel (Cheng et al., 2015), the Inventors identified the subset of mutations which fell within panel regions. Genomic coordinates were obtained, including off-target positions in noncoding regions, of the MSK-IMPACT panel from Gulhan et al. (2019). To compare against the baseline of WES, the subset of mutations from the dataset which fell within exonic regions as identified by the GENCODE project was used (Coffey et al., 2011).

TABLE 2.1

| Signature | Active Sample | Active Sample |
|---|---|---|
| 2 | 232 | 41.4% |
| 3 | 278 | 49.6% |
| 8 | 494 | 88.2% |
| 13 | 262 | 46.8% |
| 18 | 64 | 11.4% |
| 30 | 135 | 24.1% |

ScalpelSig panels were compared to a randomized baseline—the mean performance of 1000 random panels each 2.5 Mb in size. Each random panel is generated by sampling 250 unique windows from 10 Kb nonoverlapping windows across the genome. Windows with no mutations in the cohort were removed before sampling. The performance of each baseline was evaluated on the same test set as the ScalpelSig panels in each experiment.

### Evaluation on Held-Out Breast Cancer Cohort

To establish that the panels discovered by ScalpelSig are potentially applicable in the clinic, the Inventors further assessed the generalizability of ScalpelSig panels learned from one study to new samples in another. That is, ScalpelSig was evaluated on a completely held-out cohort of samples from Staaf et al. (2019). This study used the entire cohort of 560 samples from Nik-Zainal et al. (2016) to train ScalpelSig panels, and these panels were evaluated in an unseen cohort of 273 samples from Staaf et al. (2019). For this experiment, the Inventors only performed analysis with respect to signatures that resulted in panels that outperform baselines in Nik-Zainal et al. (2020) (Signatures 2, 3, 8, 13, and 18).

### Assessment of Robustness

The Inventors performed experiments to test the robustness of ScalpelSig's performance under varied conditions. First, the Inventors varied the amount of genomic material included in the panels. In these experiments the Inventors constructed ScalpelSig panels of sizes 0.1, 0.5, 1.0, 1.5, 2.0, and 2.5 Mb and evaluated their performance. Smaller panels are produced by running the ScalpelSig algorithm as described above, except during the evaluation step, mutations are counted from, for example, the topscoring 10 windows (for 0.1 Mb panels) instead of the top 250 (for 2.5 Mb panels).

Next, the Inventors varied the amount of data available for training the algorithm. In these experiments we construct ScalpelSig panels using 20%, 40%, 60%, and 80%, in addition to the default setting of 90%, of the 560 genome breast cancer cohort and evaluate their performance. For each experiment, training sets were obtained using stratified sampling as described above. Evaluation was conducted using all of the held-out samples as a test set.

The Inventors varied the threshold at which samples are considered to be active versus inactive. Experiments considered samples to be active if the signature of interest was responsible for 1%, 5%, 10%, and 20% of the total mutations in the sample, respectively. Cases where either the active or inactive class comprised fewer than 5% of samples were discarded (consistent with the protocol for our primary set of experiments) since these cases reduce the possible variation in the test set. Since class balance varied at each different setting of the activity threshold, distinct random test/train splits were sampled for each of the settings.

### Construction of a Preliminary Combined-Signature Panel

The Inventors performed an exploratory set of experiments to demonstrate that regions from individual-signature ScalpelSig panels can be combined to detect multiple signatures simultaneously. Using ScalpelSig's window scoring function at the α=0:5 setting, the Inventors took 0.5 MB of the highest scoring regions of panels designed for detection of Signatures 2, 3, 8, 13, and 18, respectively (regions for the detection of Signature 30 did not outperform baselines in the primary set of experiments and, thus, were not included). These regions were combined to form a single 2.5 MB panel and evaluated its performance for detection of those five signatures. Note that when testing for the activity of multiple signatures simultaneously, samples can no longer be neatly categorized into binary classes (i.e., a sample that is active for some signatures may be inactive for others). As a result, the stratified sampling protocol that was used to split the data into test and train sets in the individual signature panel experiments was not used. Instead uniform random sampling was used to split the data into test and train sets. As in previous experiments, 90% of the breast cancer genome cohort was used as a training set, and the remaining 10% was held out for evaluation.

### Software

ScalpelSig was implemented in R (ver. 3.6.3) (R Core Team, 2017). The code makes use of the R packages PRROC (Keilwagen et al., 2014) and pROC (Robin et al., 2011). The Inventors additionally incorporate an in-house, open-source Python implementation of the SignatureEstimation package (Huang et al., 2018).

### Results

In the desired use case for a mutational signature panel, a clinician would sequence panel regions and use them to make a judgment about the mutational signatures that have acted on the patient's genome.

Accordingly, a good panel is one where signature activity within panel regions is predictive of signature activity genome wide. The experiments measure how well a panel makes this prediction with two complementary metrics: (1) AUPR was computed for a binary classification task which asks whether a signature is substantially active in a sample given the exposure of that signature in panel regions; and (2) Spearman correlation was computed between signature exposure in panel regions and signature exposure genome wide. In the interest of clinical accessibility, this Example focuses on ScalpelSig panels that are roughly equivalent in size to the MSK-IMPACT panel, since this size is evidently practical for clinical use. However, to further characterize ScalpelSig's performance the Inventors analyzed panels at various smaller sizes. The results are contextualized by comparing ScalpelSig's performance with that of the MSK-IMPACT panel, a random panel, and whole exome sequencing. The results show that ScalpelSig improves panel accuracy substantially. In multiple cases, ScalpelSig even outperformed baselines using panels that were less than ⅔ the size of said baselines.

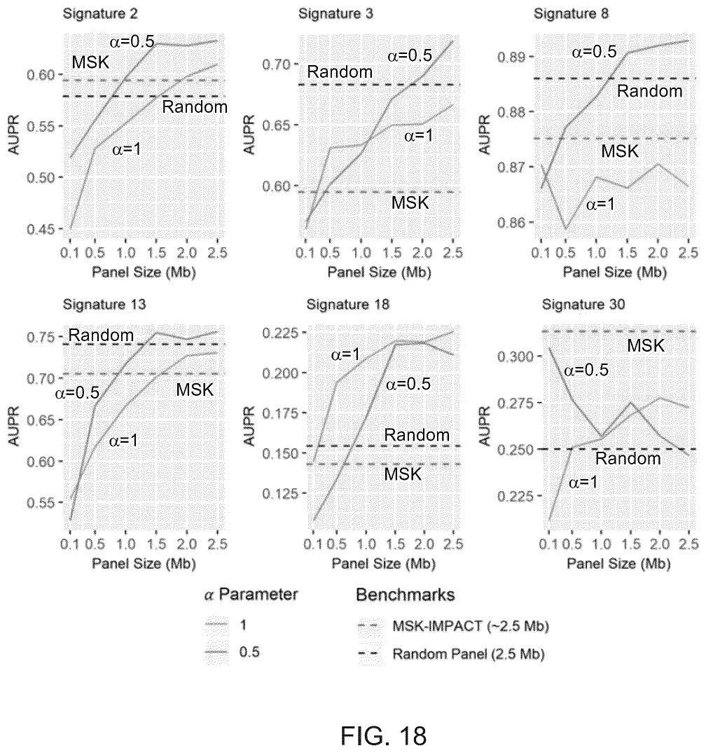

FIG. 18 shows assessment of genomic panels constructed with our framework for their ability to predict mutational signature activity at various panel sizes. Values shown are mean AUPR across 15 randomized test and train sets. Each plot tests distinct panels optimized for the detection of a particular mutational signature using two different settings of the a parameter: α=1 and α=0.5. Values are compared against a randomized baseline panel, and mean area under the precision-recall curve (AUPR) of the Memorial Sloan Kettering Integrated Mutation Profiling of Actionable Cancer Target (MSK-IMPACT) panel, for the same test sets.

Table 2.2 provides spearman correlation between exposures computed only from panel regions and exposures computed from whole genome mutation counts. Values shown are mean Spearman correlation coefficients across 15 randomized test and train sets. ScalpelSig is run with α=0.5 for all signatures. In ScalpelSig and MSK-IMPACT columns, values where fewer than half of the trials yielded p-value <0.05 are marked with an asterisk. The highest value in each row is bolded-except when most of the trials for that value are not significant.

TABLE 2.2

| | ScalpelSig | MSK-IMPACT | Random panel |
|---|---|---|---|
| Signature | (2.5 Mb) | (*2.5 Mb) | (2.5 Mb) |
| 2 | 0.3819 | 0.2695 | 0.2068 |
| 3 | 0.3883 | 0.2123* | 0.3138 |
| 8 | 0.3895 | 0.0275 | 0.1276 |
| 13 | 0.5749 | 0.4517 | 0.4580 |
| 18 | 0.1482* | 0.0309* | 0.0215 |
| 30 | 0.0569* | 0.0528* | 0.0091 |

### ScalpelSig Outperforms Baselines

Panels discovered by ScalpelSig outperform the MSK-IMPACT panel and randomized baseline on five out of the six signatures examined (Signatures 2, 3, 8, 13, and 18) in terms of both AUPR of activity/inactivity classification (FIG. 18) and Spearman correlation between panel exposures and genome-wide exposures (Table 2.2). The 2.5 Mb panels constructed using the α=1 parameterization of the window scoring function outperformed the MSK-IMPACT panel on four out of six signatures (Signatures 2, 3, 13, and 18). The 2.5 Mb panels constructed with α=0.5 outperform the α=1 setting in almost all cases, obtaining better AUPR than both the MSK-IMPACT panel and randomized baseline, with the exception of Signature 30. Classifications based on signatures extracted from WES are consistently more accurate than the other measurements, but this is to be expected since the exome (about 30 Mb) covers over ten times as much genetic material as the panels (≤2.5 Mb). Thus, the performance gap between the random baseline and WES gives a reasonable notion of how much the classification performance can be improved by simply observing more genomic material in a naive manner (the performance gap between MSK-IMPACT and WES can serve a similar function, but MSK-IMPACT performs worse than the random baseline in most cases).

Thus the efficacy of ScalpelSig panels is demonstrated by their ability to partially bridge this performance gap. Table 2.3 provides comparison of ScalpelSig (α=0.5; 2.5 Mb) panels to whole exome (30 Mb) and random panel (2.5 Mb) benchmarks for five out of six examined signatures. The right-most column gives the percentage of the performance gap between WES and the random panel that is bridged by our method.

TABLE 2.3

| | Area under the precision-recall curve | | | |
|---|---|---|---|---|
| Signature | Whole exome | Random panel | ScalpelSig | Gap bridged |
| 2 | 0.7959 | 0.5785 | 0.6330 | 25.1% |
| 3 | 0.8727 | 0.6826 | 0.7191 | 19.1% |
| 8 | 0.9338 | 0.8860 | 0.8929 | 14.5% |
| 13 | 0.9370 | 0.7408 | 0.7562 | 7.9% |
| 18 | 0.3027 | 0.1542 | 0.2111 | 38.3% |

By this metric, ScalpelSig's increase in performance over the baseline panels ranged from moderate to sizable, bridging at least 7.9% and at most 38.3% of the performance gap between the randomized panel and whole exome sequencing (Table 2.3). If one assumes, solely for the purpose of ballpark estimation, that performance scales linearly with addition of exomic regions to the panel, these improvements represent an increase in performance proportional to the addition of between 2.1 and 10.5 Mb of exomic material to the 2.5 Mb baseline. As noted above, the α=0.5 parameterization is designed to obtain results that better generalize outside of the training set by lowering the impact of windows where just a few active samples have very high signature activity and favoring windows where a large number of active samples have moderately high activity. The improved performance of α=0.5 over α=1 suggests the necessity and effectiveness of this parameterization.

The MSK-IMPACT panel performs worse than the random baseline on all examined signatures except Signatures 2 and 30. The MSK-IMPACT panel was designed for the detection of common driver mutations, so it follows that the mutations it captures have a different distribution than the distribution of mutations over the whole genome (and consequently, the signatures obtained from its captured mutations may be inconsistent with genome-wide signatures). It is additionally noted that the random panel benchmark is in actuality given by the mean performance of 1000 panels with randomly selected windows.

To assess the generalizability of the panels designed by ScalpelSig, the Inventors evaluated its performance on a completely held-out dataset. To identify a single panel per signature, we trained ScalpelSig on the whole cohort of 560 breast cancer genomes described above (Nik-Zainal et al., 2016) and evaluated the resulting panels using a new cohort of 237 breast cancer genomes as a test set from Staaf et al. (2019). The results show that ScalpelSig continued to outperform the MSK-IMPACT panel in this setting, both in terms of Spearman correlation and AUPR (Table 2.4, below). This provides evidence that the improved performance of ScalpelSig panels generalizes beyond the initial dataset used in this Example.

Table 2.4 provides evaluation of ScalpelSig (a=0:5) and MSK-IMPACT on a completely held-out dataset of 237 breast cancer genomes from Staaf et al. (2019). Each row reports the results of a single ScalpelSig panel, trained on all 560 samples from the previous dataset. Spearman correlations with p-value $0.05 are not shown. The highest values in each row for each of the two evaluation metrics (Spearman and AUPR) are bolded

TABLE 2.4

| Spearman correlation | | | AUPR | |
|---|---|---|---|---|
| Signature | ScalpelSig | MSK-IMPACT | ScalpelSig | MSK-IMPACT |
| 2 | 0.3437 | 0.1804 | 0.3844 | 0.2900 |
| 3 | 0.5048 | 0.4749 | 0.9321 | 0.9137 |
| 8 | 0.4275 | 0.2004 | 0.9406 | 0.9195 |
| 13 | 0.5365 | 0.3276 | 0.7318 | 0.6837 |
| 18 | — | — | 0.1458 | 0.0298 |

### Robustness of Performance

Below, several experiments are presented to demonstrate that ScalpelSig maintains a strong level of performance, even outside of the canonical conditions featured in the study thus far. The amount of genomic material that is included in the panel, the amount of data available for training, and the threshold at which samples are considered to be active versus inactive, were varied. The robust performance of ScalpelSig in these various settings is evidence that the method may find effective application outside of the controlled setting of this study.

### Performance at Smaller Panel Sizes

The Inventors investigated how performance of designed panels changed at different panel sizes, moving beyond our initial focus on the 2.5 Mb panel size used by MSKIMPACT. While one may expect that the more genomic material is sampled, the better performance should become (as the number of observed mutations approaches the whole genome mutation count as the panel gets larger), there are a few exceptions to this expectation. Firstly, it is noted that panel performance is not a monotonically increasing function of panel size (FIG. 18). This phenomenon is unintuitive, but in fact observing additional genome regions is not guaranteed to increase accuracy and may even decrease it. Individual genome windows may have mutation distributions which differ starkly from the genome-wide distribution, due to variation in nucleotide composition and other factors. Adding such windows to a panel could produce a mutation distribution that misrepresents the genome-wide distribution, despite containing a greater number of mutations. This misleading distribution would result in a false impression of signature activity. Thus it is possible for performance to decline with the addition of certain windows.

It is further observed that panels constructed with α=0.5 for Signatures 2, 13, and 18 all appear to plateau in their performance before the 2.5 Mb panel size is reached. This plateau seems to occur at 1.5 Mb for Signatures 2 and 13 and at 2.0 Mb for Signature 18. This indicates that in some cases, panels significantly smaller than those presently in clinical use may be sufficient to achieve the full performance boost provided by the method of the present embodiments. Recall that the panel is formed from the highest-scoring windows. As the panel gets bigger, progressively lower-scoring windows are added, so it follows that the performance increase might plateau.

Performance with Limited Training Data

To investigate the amount of data required to effectively train ScalpelSig, the Inventors performed experiments in which we varied the amount of training data. While using 90% of the cohort (504 samples) as training data were the default setting, the Inventors additionally ran ScalpelSig using 80%, 60%, 40%, and 20% of the cohort (448, 336, 224, and 112 samples, respectively) as a training set and observed only slight declines in performance (Table 2.5, below). Indeed, even at the lowest setting (using 20% of the cohort as training data rather than the usual 90%) ScalpelSig continues to outperform MSK-IMPACT in terms of both Spearman correlation and AUPR for Signatures 2, 3, 8, and 13 (see Table 2.2 and FIG. 18 for the performance of MSK-IMPACT). These results demonstrate the stability of the performance demonstrated above, but they also demonstrate that ScalpelSig may be effective even when applied to cancer types that have substantially fewer whole-genome samples available. This broadens the potential use cases of ScalpelSig, furthering the goal of expanding clinical access to MSA.

Table 2.5 provides spearman correlation between exposures computed using panel regions and exposures computed from whole genome mutation counts, as well as AUPR for signature activity prediction, for panels constructed by ScalpelSig using various amounts of training data. For each row, 15 randomized training sets were obtained using stratified sampling as detailed in the Section 3. The size of the training sets for each row is indicated in the Training Data column. The ScalpelSig algorithm was run separately on each of the training sets and evaluated using all of the held-out samples. Values shown are mean Spearman correlation coefficients and mean AUPR across the randomized trials. Spearman values where fewer than half of the trials yielded p-value <0.05 are marked with an asterisk.

TABLE 2.5

| Signature | % Training data | Spearman | AUPR |
|---|---|---|---|
| 2 | 90 | 0.3819 | 0.6330 |
| | 80 | 0.4006 | 0.6462 |
| | 60 | 0.3850 | 0.6199 |
| | 40 | 0.3975 | 0.6302 |
| | 20 | 0.3639 | 0.6197 |
| 3 | 90 | 0.3883 | 0.7191 |
| | 80 | 0.3830 | 0.6984 |
| | 60 | 0.3635 | 0.6888 |
| | 40 | 0.3572 | 0.7002 |
| | 20 | 0.3722 | 0.7025 |
| 8 | 90 | 0.3895 | 0.8929 |
| | 80 | 0.3479 | 0.8834 |
| | 60 | 0.3600 | 0.891 |
| | 40 | 0.3434 | 0.8891 |
| | 20 | 0.3400 | 0.8978 |
| 13 | 90 | 0.5749 | 0.7562 |
| | 80 | 0.5467 | 0.7433 |
| | 60 | 0.5393 | 0.7405 |
| | 40 | 0.5596 | 0.7372 |
| | 20 | 0.5416 | 0.7409 |
| 18 | 90 | 0.1482* | 0.2111 |
| | 80 | 0.0792* | 0.158 |
| | 60 | 0.0749* | 0.1612 |
| | 40 | 0.0792* | 0.141 |
| | 20 | 0.0965* | 0.163 |
| 30 | 90 | 0.0569* | 0.2499 |
| | 80 | 0.1032* | 0.2493 |
| | 60 | 0.0787* | 0.2567 |
| | 40 | 0.06884* | 0.2479 |
| | 20 | 0.0888 | 0.254 |

### Performance Across Varied Activity Thresholds

ScalpelSig separates genomes into binary active and inactive categories for each signature. The threshold of signature activity for this categorization is a parameterization choice. In this Example, the activity threshold was set to be 5% (i.e., if a signature contributes 5% or more of the mutations in a genome, that genome is considered to be active for that signature). To show the robustness of the algorithm to different parameterization choices, the Inventors experimented with other thresholds. The Inventors tested multiple values for the activity threshold (1%, 5%, 10%, 20%) and show these results in Table 2.6.

Table 2.6 provides spearman correlation between exposures computed using panel regions and exposures computed from whole genome mutation counts, for panels constructed using various thresholds for activity classification. The Activity Threshold column gives the required percentage of mutations contributed by a signature for a sample to be considered active. That is, in the first row samples were classified as active if at least 1% of their mutations were contributed by Signature 2. In the second row samples were considered active if at least 5% (the default for this study) came from Signature 2, etc. For the ScalpelSig columns, and the MSK-IMPACT column, values shown are mean Spearman correlation coefficients across 15 randomized test and train sets. Different random test/train splits were sampled for each row. Cases where either the active or inactive class comprised fewer than 5% of the samples were discarded, since these cases reduce the possible variation in the test set. Spearman values where fewer than half of the trials yielded p-value <0.05 are marked with an asterisk. ScalpelSig was applied with α=0.5.

TABLE 2.6

| | Activity | Active | | |
|---|---|---|---|---|
| Signature | threshold | samples | ScalpelSig | MSK-IMPACT |
| 2 | 1 | 88.4 | 0.3965 | 0.2688 |
| | 5 | 41.4 | 0.3819 | 0.2695 |
| | 10 | 23.6 | 0.4171 | 0.3193 |
| | 20 | 10.7 | 0.4532 | 0.2903 |
| 3 | 1 | 66.4 | 0.3832 | 0.1597* |
| | 5 | 49.6 | 0.3883 | 0.2123* |
| | 10 | 38.4 | 0.3775 | 0.2733 |
| | 20 | 28.4 | 0.4020 | 0.2273* |
| 8 | 1 | 95.5 | — | — |
| | 5 | 88.2 | 0.3895 | 0.0275 |
| | 10 | 73.6 | 0.2815 | 0.0447* |
| | 20 | 27.0 | 0.3035 | 0.1000* |
| 13 | 1 | 92.0 | 0.5378 | 0.4234 |
| | 5 | 46.8 | 0.5749 | 0.4517 |
| | 10 | 73.6 | 0.4905 | 0.4636 |
| | 20 | 27.0 | 0.5125 | 0.4671 |
| 18 | 1 | 32.9 | 0.0803* | 0.0213* |
| | 5 | 11.4 | 0.1482* | 0.0309* |
| | 10 | 4.6 | — | — |
| | 20 | 0.5 | — | — |
| 30 | 1 | 63.0 | 0.1255* | 0.1163* |
| | 5 | 24.1 | 0.0569* | 0.0528* |
| | 10 | 3.5 | — | — |
| | 20 | 0.2 | | |

It was found that ScalpelSig maintains a strong performance across these varied settings-ScalpelSig outperforms MSK-IMPACT across the board for Signatures 2, 3, 8, and 13. The default activity threshold of 5% generally performed the best, but this was not always the case. For example, on Signature 2 the stricter thresholds of 10% and 20% achieved a moderate increase in performance compared to the default. These suggest that the method can obtain even higher performance by tuning the activity threshold parameter for specific use cases.

An additional detail of Table 2.6 is that different test and train sets were sampled in each row. This was done since the class balance changes as the activity threshold is varied (see the Active Samples column), and the Inventors used stratified sampling to guarantee that the class balance of test sets is equivalent to the overall cohort. As a consequence, the values for the MSK-IMPACT panel vary across experiments despite the fact that the observed genome regions remain the same.

### A Combined-Signature Panel

The present designed a distinct panel for each individual signature of interest. However, the method of the present embodiments can also detect the activity of multiple signatures simultaneously from the same genomic regions. The Inventors performed a set of experiments to demonstrate that regions from individual-signature ScalpelSig panels can be combined to detect multiple signatures simultaneously. The Inventors took 0.5 MB of the highest scoring regions of panels designed for detection of Signatures 2, 3, 8, 13, and 18, respectively. These regions weer combined to form a single 2.5 MB panel and observed its performance for detecting the same five signatures. The results are provided in Table 2.7.

Table 2.7 provides results from assessing genomic panels designed to detect multiple signatures simultaneously. The panels were constructed by combining windows from ScalpelSig (α=0.5) panels for individual signature detection. No windows for detection of Signature 30 were incorporated into the panel, and we did not evaluate the combined panel's performance at detecting Signature 30, since the ScalpelSig panels that were individually optimized for Signature 30 detection did not outperform baselines. Values shown are median AUPR and Spearman correlation across 15 randomized test and train sets. We used uniform random sampling to split the data into test and train sets. This means that there was more variance in test sets of the combined panel experiments, which likely accounts for the differences in the MSK-IMPACT scores in comparison to the individual signature panel experiments.

TABLE 2.7

| | Spearman correlation | | AUPR | |
|---|---|---|---|---|
| Signature | ScalpelSig | MSK-IMPACT | ScalpelSig | MSK-IMPACT |
| 2 | 0.3865 | 0.3162 | 0.6232 | 0.6091 |
| 3 | 0.3700 | 0.1698 | 0.6894 | 0.5790 |
| 8 | 0.2948 | 0.0507 | 0.8918 | 0.8900 |
| 13 | 0.4796 | 0.4401 | 0.7270 | 0.6962 |
| 18 | 0.1102 | 0.0675 | 0.1723 | 0.1193 |

Overall, the combined panel outperforms MSK-IMPACT. This it indicates that the individual panels are indeed specialized for the detection of their respective signatures. That is, the regions detected in the individual-panel experiments are not interchangeable-mixing and matching incurs a substantial penalty to performance. This suggests that the genome regions detected by the method of the present embodiments may have biologically meaningful differences in terms of their active mutational processes.

### DISCUSSION

This Example presented a method that designs genomic panels optimized for the detection of mutational signature activity. ScalpelSig takes as input a mutational signature and a set of training tumor genomes and learns genome regions in which mutations are highly indicative of signature activity.

ScalpelSig was trained on breast cancer data to obtain panels optimized for the detection of six respective mutational signatures. It was found that in five out of six examined signatures, ScalpelSig panels outperform the commonly studied MSK-IMPACT panel and a random baseline. The increased accuracy of the panels designed by ScalpelSig according to the present embodiments is substantial even compared to whole exome sequencing (Table 2.3), which uses over 10 times as much genomic material as targeted panel sequencing. The results show that ScalpelSig panels bridge significant portions of the performance disparity between the baseline panels and the whole exome benchmark. In the most favorable case, this performance increase is roughly proportional to the addition of 10.5 Mb of exonic material to the baseline panels. It was found that the performance of ScalpelSig is robust under a variety of parameterizations. For four of six examined signatures, panels smaller than 2.5 Mb are sufficient to obtain all or most of the demonstrated performance increase. Furthermore, the panels designed by ScalpelSig maintain strong performance even when the amount of training data is significantly reduced. These results demonstrate that the performance increase afforded by the method of the present embodiments can generalize beyond the conditions of the study presented in this Example. For example, the method can be used in other cancer types that have less training data available.

Of the signatures investigated in this Example, Signature 3 is arguably the most exciting as a clinically actionable biomarker. Signature 3 has been found to be correlated with biomarkers of homologous recombination repair deficiency (Nik-Zainal et al., 2016; Davies et al., 2017; Polak et al., 2017; Gulhan et al., 2019), which is a biomarker for PARP inhibitor therapy (Farmer et al., 2005). Recently, Gulhan et al. (2019) developed an algorithm for improved detection of Signature 3 from MSK-IMPACT panel data. The results demonstrate that ScalpelSig panels confer a substantial increase in accuracy for detection of Signature 3 compared to MSKIMPACT when using standard methods for signature detection. Relative to MSK-IMPACT, ScalpelSig panels give an 83% increase in Spearman correlation coefficient between Signature 3 activity in panel regions and genome-wide Signature 3 activity. It is noted that when the panels were evaluated on a held-out dataset, the improvement for Signature 3 was less pronounced. The algorithm used in Gulhan et al (2019) can be trained using ScalpelSig panel data to achieve a synergistic boost in accuracy for detection of Signature 3. Combining the two approaches can be a boon for detecting HR deficiency in the clinic.

Other methods for inferring signature exposures and active signatures can be used. Those include Huang et al. (2018)'s method for automatically detecting which signatures are active in each sample with confidence. It is appreciated that the method of the present embodiments can employ other parameterizations, such as, but not limited to, hyperparameter tuning using cross-validation. The machine learning procedure of the present embodiments trained for other cancer types and signatures.

### Annex

### Mathematical Derivation

The Mix model is parameterized by θ=(w, π, e), where w denotes the cluster probabilities, π is a collection of signature exposures for each cluster, and e represents the signatures that are shared among all clusters. The parameters that are relevant for cluster [symbol] are denote by [symbol] =( [symbol] , e). The model's hyper-parameters are L, K, which denote the number of clusters and the number of signatures. In addition, N denotes the number of samples, and M denotes the number of mutation categories. In the derivation below, n, [symbol] , k, m are the indices that run on [N], [L], [K], [M] respectively. The observed mutation data are denoted as O=O¹, . . . , O^{N} where:

$${O}^{i} = {o}_{1}^{i} … {o}_{{T}_{i}}^{i}$$

are the mutations of sample i. The hidden cluster and signature identity data are denoted as H=C, Z where C=c¹ . . . c^{N} are the clusters of each of the samples, and Z=Z¹ . . . . Z^{N}, where

$${Z}^{i} = {z}_{1}^{i} … {z}_{N}^{i}$$

are the signatures that underlie each mutation in each sample.

For clarity of presentation, indices are omitted, when possible, and general variables for cluster, signature and mutation are denote by w, z, and o, respectively. Formally,

$$\text{} \begin{matrix}∀ ℓ & {w}_{ℓ} = Pr [ {c}^{n} = ℓ \vert θ ] , {\sum }_{ℓ} {w}_{ℓ} = 1\end{matrix}$$

$$∀ ℓ , k \text{} \qquad {π}_{k}^{ℓ} = Pr [ {z}_{t}^{n} = k \vert {w}^{n} = ℓ , θ ] = Pr [ z = k \vert {θ}^{ℓ} ] , {\sum }_{k} {π}_{k}^{ℓ} = 1$$

$$∀ k , m \qquad {e}_{k} ( m ) = Pr [ {o}_{t}^{n} = m \vert {z}_{t}^{n} = k , θ ] .$$

For convenience, for every possible choice of hidden data H, the following notations are used:

$$\text{} \begin{matrix}∀ ℓ & {W}_{ℓ} ( H ) = \vert \{ n \vert {w}^{n} = ℓ \} \vert\end{matrix}$$

$$\text{} \begin{matrix}∀ ℓ , k & {A}_{k}^{ℓ} ( H ) = \vert \{ ( n , t ) \vert {w}^{n} = ℓ , {z}_{t}^{n} = k \} \vert\end{matrix}$$

$$\text{} \begin{matrix}∀ ℓ , m & {E}_{k} ( m , H ) = \vert \{ ( n , t ) \vert {z}_{t}^{n} = k , {o}_{t}^{n} = m \} \vert\end{matrix}$$

The log likelihood is given by:

$$log Pr [ H , O \vert θ ] = {\sum }_{ℓ} {W}_{ℓ} ( H ) log {w}_{ℓ} + {\sum }_{ℓ} {\sum }_{k} {A}_{k}^{ℓ} ( H ) log {π}_{k}^{ℓ} + {\sum }_{k} {\sum }_{m} {E}_{k} ( m , H ) log {e}_{k} ( m )$$

The Q function to maximize (expected complete log likelihood) is given by:

$$Q ( θ \vert {θ}^{0} ) = {\sum }_{H} Pr [ H \vert O , {θ}^{0} ] log Pr [ H , O \vert θ ] = {\sum }_{ℓ} {\sum }_{H} Pr [ H \vert O , {θ}^{0} ] {W}_{ℓ} ( H ) log {w}_{ℓ} + {\sum }_{ℓ} {\sum }_{k} {\sum }_{H} Pr [ H \vert O , {θ}^{0} ] {A}_{k}^{ℓ} ( H ) log {π}_{k}^{ℓ} + {\sum }_{k} {\sum }_{m} {\sum }_{H} Pr [ H \vert O , {θ}^{0} ] {E}_{k} ( m , H ) log {e}_{k} ( m ) = {\sum }_{ℓ} {W}_{ℓ} log {w}_{ℓ} + {\sum }_{ℓ} {\sum }_{k} {A}_{k}^{ℓ} log {π}_{k}^{ℓ} + {\sum }_{k} {\sum }_{m} {E}_{k} ( m ) log {e}_{k} ( m )$$

$$where$$

$${W}_{ℓ} = {\sum }_{H} Pr [ H \vert O , {θ}^{0} ] {W}_{ℓ} ( H )$$

$${A}_{k}^{ℓ} = {\sum }_{H} Pr [ H \vert O , {θ}^{0} ] {A}_{k}^{ℓ} ( H )$$

$${E}_{k} ( m ) = {\sum }_{H} Pr [ H \vert O , {θ}^{0} ] {E}_{k} ( m , H )$$

It will now be shown that the Q function is maximized for the M-step given in the Methods under the following restrictions:

$${\sum }_{ℓ} {w}_{ℓ} = 1 , ∀ ℓ {\sum }_{k} {π}_{k}^{ℓ} = 1 , ∀ k {\sum }_{m} {e}_{k} ( m ) = 1$$

The following probabilities will be used:

$$Pr [ z = k , o = m \vert {θ}^{ℓ} ] = Pr [ z = k \vert {θ}^{ℓ} ] Pr [ o = m \vert z = k , {θ}^{ℓ} ] = {π}_{k}^{ℓ} {e}_{k} ( m )$$

$$Pr [ o = m \vert {θ}^{ℓ} ] = {\sum }_{k} Pr [ z = k , o = m \vert {θ}^{ℓ} ] = {\sum }_{k} {π}_{k}^{ℓ} {e}_{k} ( m )$$

$$Pr [ z = k \vert o = m , {θ}^{ℓ} ] = \frac{Pr [ z = k \vert {θ}^{ℓ} ] Pr [ o = m \vert z = k , {θ}^{ℓ} ]}{Pr [ o = m \vert {θ}^{ℓ} ]} = \frac{{π}_{k}^{ℓ} {e}_{k}^{( m )}}{{\sum }_{k} {π}_{{k}^{′}}^{ℓ} {e}_{{k}^{′}} ( m )}$$

$$Pr [ c = ℓ , O \vert θ ] = Pr [ c = ℓ \vert θ ] Pr [ O \vert {θ}^{ℓ} ] = {w}_{ℓ} {\prod }_{t} Pr [ {o}_{t} \vert {θ}^{ℓ} ]$$

$$r [ O \vert θ ] = {\sum }_{ℓ} Pr [ c = ℓ \vert θ ] Pr [ O \vert {θ}^{ℓ} ] = {\sum }_{ℓ} {w}_{ℓ} {\prod }_{t} Pr [ {o}_{t} \vert {θ}^{ℓ} ]$$

$$Pr [ {c}^{ℓ} \vert O , θ ] = \frac{Pr [ c = ℓ \vert θ ] Pr [ O \vert {θ}^{ℓ} ]}{Pr [ O \vert θ ]} = \frac{{w}_{ℓ} {\prod }_{t} Pr [ {o}_{t} \vert {θ}^{ℓ} ]}{{\sum }_{{ℓ}^{′}} {w}_{{ℓ}^{′}} {\prod }_{t} Pr [ {o}_{ℓ} \vert {θ}^{{ℓ}^{t}} ]}$$

Below is a preferred procedure suitable for computing the variables. The 0 index is omitted from θ₀, and I* is used as the indicator of some outcome:

$$\begin{matrix}{W}_{ℓ} = {\sum }_{H} Pr [ H \vert O , θ ] {W}_{ℓ} ( H ) \\ = {\sum }_{n} {\sum }_{{H}^{n}} Pr [ {H}^{n} \vert {O}^{n} , θ ] {I}_{{c}^{n} = ℓ} \\ = {\sum }_{n} {\sum }_{{Z}^{n}} Pr [ {c}^{n} = ℓ , {Z}^{n} \vert {O}^{n} , θ ] {I}_{{c}^{n} = ℓ} \\ = {\sum }_{n} Pr [ {c}^{n} = ℓ \vert {O}^{n} , θ ]\end{matrix}$$

$$\begin{matrix}{A}_{k}^{ℓ} = {\sum }_{H} Pr [ H \vert O , θ ] {A}_{k}^{ℓ} ( H ) \\ = {\sum }_{n} {\sum }_{{H}^{n}} Pr [ {H}^{n} \vert {O}^{n} , θ ] {A}_{k}^{ℓ} ( H ) {I}_{{c}^{n} = ℓ} \\ = {\sum }_{n} {\sum }_{{Z}^{n}} Pr [ {c}_{n} = ℓ , {Z}^{n} \vert {O}^{n} , θ ] {A}_{k}^{ℓ} ( {Z}^{n} ) \\ = {\sum }_{n} Pr [ {c}_{n} = ℓ \vert {O}^{n} , θ ] {\sum }_{{Z}^{n}} Pr [ {Z}^{n} \vert {O}^{n} , {θ}^{ℓ} ] {A}_{k}^{ℓ} ( {Z}^{n} ) \\ = {\sum }_{n} Pr [ {c}_{n} = ℓ \vert {O}^{n} , θ ] {\sum }_{{Z}^{n}} Pr [ {Z}^{n} \vert {O}^{n} , {θ}^{ℓ} ] ( {\sum }_{t} {I}_{{z}_{t}^{n} = k} ) \\ = {\sum }_{n} Pr [ {c}_{n} = ℓ \vert {O}^{n} , θ ] {\sum }_{t} {\sum }_{{Z}^{n}} Pr [ {Z}^{n} \vert {O}^{n} , {θ}^{ℓ} ] {I}_{{z}_{t}^{n} = k} \\ = {\sum }_{n} Pr [ {c}_{n} = ℓ \vert {O}^{n} , θ ] {\sum }_{t} {\sum }_{{Z}^{n} \vert {z}_{t}^{n} = k} Pr [ {Z}^{n} \vert {O}^{n} , {θ}^{ℓ} ] \\ = {\sum }_{n} Pr [ {c}_{n} = ℓ \vert {O}^{n} , θ ] {\sum }_{t} Pr [ {z}_{t}^{n} = k \vert {O}^{n} , {θ}^{ℓ} ] \\ = {\sum }_{n} Pr [ {c}_{n} = ℓ \vert {O}^{n} , θ ] {\sum }_{t} Pr [ z = k \vert o = {o}_{t}^{n} , {θ}^{ℓ} ]\end{matrix}$$

$$\begin{matrix}{E}_{k} ( m ) = {\sum }_{H} Pr [ H \vert O , θ ] {E}_{k} ( m , H ) \\ = {\sum }_{n} {\sum }_{{H}^{n}} Pr [ {H}^{n} \vert {O}^{n} , θ ] {E}_{k} ( m , {H}^{n} ) \\ = {\sum }_{n} {\sum }_{ℓ} {\sum }_{{H}^{n} \vert {c}^{n} = ℓ} Pr [ {H}^{n} \vert {O}^{n} , θ ] {E}_{k} ( m , {H}^{n} ) \\ = {\sum }_{n} {\sum }_{ℓ} {\sum }_{{Z}^{n}} Pr [ {c}^{n} = ℓ , {Z}^{n} \vert {O}^{n} , θ ] {E}_{k} ( m , {Z}^{n} ) \\ = {\sum }_{n} {\sum }_{ℓ} Pr [ {c}_{n} = ℓ \vert {O}^{n} , θ ] {\sum }_{{Z}^{n}} Pr [ {Z}^{n} \vert {O}^{n} , {θ}^{ℓ} ] {E}_{k} ( m , {Z}^{n} ) \\ = {\sum }_{n} {\sum }_{ℓ} Pr [ {c}_{n} = ℓ \vert {O}^{n} , θ ] {\sum }_{{Z}^{n}} Pr [ {Z}^{n} \vert {O}^{n} , {θ}^{ℓ} ] ( {\sum }_{t} {I}_{{z}_{t}^{n} = k , {o}_{t}^{n} = m} ) \\ = {\sum }_{n} {\sum }_{ℓ} Pr [ {c}_{n} = ℓ \vert {O}^{n} , θ ] {\sum }_{ℓ} {\sum }_{{Z}^{n}} Pr [ {Z}^{n} \vert {O}^{n} , {θ}^{ℓ} ] {I}_{{z}_{t}^{n} = k , {o}_{t}^{n} = m} \\ = {\sum }_{n} {\sum }_{ℓ} Pr [ {c}_{n} = ℓ \vert {O}^{n} , θ ] {\sum }_{t \vert {o}_{t}^{n} = m} {\sum }_{{Z}^{n} \vert {z}_{t}^{n} = k} Pr [ {Z}^{n} \vert {O}^{n} , {θ}^{ℓ} ] \\ = {\sum }_{n} {\sum }_{ℓ} Pr [ {c}_{n} = ℓ \vert {O}^{n} , θ ] {\sum }_{t \vert {o}_{t}^{n} = m} Pr [ z = k \vert o = m , {θ}^{ℓ} ] \\ = {\sum }_{n} {\sum }_{ℓ} Pr [ {c}_{n} = ℓ \vert {O}^{n} , θ ] \vert \{ t \vert {o}_{t}^{n} = m \} \vert Pr [ z = k \vert o = m , {θ}^{ℓ} ]\end{matrix}$$

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES FOR EXAMPLE 1

[1] Thomas, H., et al.: Mechanisms underlying mutational signatures in human cancers. Nature Reviews Genetics 15(9), 585-598 (2014). doi:10.1038/nrg3729
[2] Anthony, T., et al.: Endogenous DNA Damage as a Source of Genomic Instability in Cancer. Cell 168(4), 644-656 (2017). doi:10.1016/j.cell.2017.01.002
[3] Ludmil, B. A., et al.: Signatures of mutational processes in human cancer. Nature 500(7463), 415-421 (2013). doi:10.1038/nature12477
[4] Ludmil, B. A., et al. : Deciphering Signatures of Mutational Processes Operative in Human Cancer. Cell Reports 3(1), 246-259 (2013). doi:10.1016/j.celrep.2012.12.008
[5] Serena, N.-Z., et al.: Mutational Processes Molding the Genomes of 21 Breast Cancers. Cell 149(5), 979-993 (2012). doi:10.1016/j.cell.2012.04.024
[6] Ludmil, B. A., et al. : Mutational signatures associated with tobacco smoking in human cancer. Science 354(6312), 618-622 (2016). doi:10.1126/science.aag0299
[7] Navnath, S. G., et al.: DNA repair targeted therapy: The past or future of cancer treatment?Pharmacology Therapeutics 160, 65-83 (2016). doi:10.1016/j.pharmthera.2016.02.003
[8] Anchit, K.: DNA Damage in Cancer Therapeutics: A Boon or a Curse?Cancer Research 75(11), 2133-2138 (2015). doi:10.1158/0008-5472.can-14-3247
[9] Kent, W. M., et al.: DNA Damage and Repair Biomarkers of Immunotherapy Response. Cancer Discovery 7(7), 675-693 (2017). doi:10.1158/2159-8290.cd-17-0226
[10] Mark, J. O.: Targeting the DNA Damage Response in Cancer. Molecular Cell 60(4), 547{560 (2015). doi:10.1016/j.molcel.2015.10.040
[11] Helen, D., et al.: HRDetect is a predictor of BRCA1 and BRCA2 deficiency based on mutational signatures. Nature Medicine 23(4), 517{525 (2017). doi:10.1038/nm.4292
[12] Hannah, F., et al.: Targeting the DNA repair defect in BRCA mutant cells as a therapeutic strategy. Nature 434(7035), 917-921 (2005). doi:10.1038/nature03445
[13] Kyle, C., et al.: Mutation signatures reveal biological processes in human cancer. bioRxiv, 036541 (2016). doi:10.1101/036541
[14] Andrej, F., et al.: EMu: probabilistic inference of mutational processes and their localization in the cancer genome. Genome Biology 14(4), 1-10 (2013). doi:10.1186/gb-2013-14-4-r39
[15] Jaegil, K., et al.: Somatic ERCC2 mutations are associated with a distinct genomic signature in urothelial tumors. Nature Genetics 48(6), 600-606 (2016). doi:10.1038/ng.3557
[16] Rafael, A. R., et al. : signeR: an empirical Bayesian approach to mutational signature discovery. Bioinformatics 33(1), 8-16 (2016). doi:10.1093/bioinformatics/btw572
[17] Xiaoqing, H., et al.: Detecting presence of mutational signatures in cancer with confidence. Bioinformatics (Oxford and England) (2017). doi:10.1093/bioinformatics/btx604
[18] Rachel, R., et al.: deconstructSigs: delineating mutational processes in single tumors distinguishes DNA repair deficiencies and patterns of carcinoma evolution. Genome Biology 17(1), 31 (2016). doi:10.1186/s13059-016-0893-4
[19] Blokzijl, F., et al.: MutationalPatterns: comprehensive genome-wide analysis of mutational processes. Genome Medicine 10, 33 (2018). doi:10.1186/s13073-018-0539-0
[20] Funnell, T., et al.: Integrated single-nucleotide and structural variation signatures of DNA-repair deficient human cancers. PLOS Computational Biology 15(2): e1006799 (2019)
[21] Yuichi, S., et al.: A Simple Model-Based Approach to Inferring and Visualizing Cancer Mutation Signatures. PLOS Genetics 11(12), 1005657 (2015). doi:10.1371/journal.pgen.1005657
[22] Wojtowicz, D., et al.: Hidden Markov models lead to higher resolution maps of mutation signature activity in cancer. Genome Medicine 11, 49 (2019). doi:10.1186/s13073-019-0659-1
[23] Robinson, W., et al.: Modeling clinical and molecular covariates of mutational process activity in cancer. Bioinformatics 35(14), 492-500 (2019). doi:10.1093/bioinformatics/btz340
[24] Gulhan, D. C., et al.: Detecting the mutational signature of homologous recombination deficiency in clinical samples. Nature Genetics 51, 912-919 (2019). doi:10.1038/s41588-019-0390-2
[25] Blei, D. M., et al.: Latent dirichlet allocation. J. Mach. Learn. Res. 3, 993-1022 (2003). doi:10.1162/jmlr.2003.3.4-5.993
[26] Cheng, D. T., et al.: Memorial sloan kettering-integrated mutation profiling of actionable cancer targets (msk-impact): a hybridization capture-based next-generation sequencing clinical assay for solid tumor molecular oncology. The Journal of molecular diagnostics 17(3), 251-264 (2015). doi:10.1016/j.jmoldx.2014.12.006
[27] Zehir, A., et al.: Mutational landscape of metastatic cancer revealed from prospective clinical sequencing of 10,000 patients. Nature medicine 23(6), 703 (2017). doi:10.1038/nm.4333
[28] Nik-Zainal, S., et al.: Landscape of somatic mutations in 560 breast cancer whole-genome sequences. Nature 534(7605), 47 (2016). doi:10.1038/nature17676
[29] Tomczak, K., et al.: The cancer genome atlas (tcga): an immeasurable source of knowledge. Contemporary oncology 19(1A), 68 (2015). doi:10.5114/wo.2014.47136
[30] Staaf, J., et al.: Whole-genome sequencing of triple-negative breast cancers in a population-based clinical study. Nature medicine 25(10), 1526-1533 (2019). doi:10.1038/s41591-019-0582-4
[31] Rizvi, H. et al.: Molecular determinants of response to anti-programmed cell death (pd)-1 and anti-programmed death-ligand 1 (pd-li) blockade in patients with non-small-cell lung cancer profiled with targeted next-generation sequencing. Journal of Clinical Oncology 36(7), 633 (2018). doi:10.1200/JCO.2017.75.3384
[32] Gao, J., et al.: The cbio cancer genomics portal: an open platform for exploring multidimensional cancer genomics data. Cancer discovery, 401-404 (2012). doi:10.1158/2159-8290.CD-12-0095
[33] Gao, J., et al.: Integrative analysis of complex cancer genomics and clinical profiles using the cbioportal. Sci. Signal (2013). doi:10.1126/scisignal.2004088
[34] Samstein, R. M., et al.: Tumor mutational load predicts survival after immunotherapy across multiple cancer types. Nature genetics 51(2), 202-206 (2019)
[35] Pedregosa, F., et al.: Scikit-learn: Machine learning in Python. Journal of Machine Learning Research 12, 2825-2830 (2011)
[36] Virtanen, P., et al.: SciPy 1.0-Fundamental Algorithms for Scientific Computing in Python. arXiv e-prints (2019). 1907.10121
[37]Köster, J., et al.: Snakemake—a scalable bioinformatics work ow engine. Bioinformatics 28(19), 2520-2522 (2012). doi:10.1093/bioinformatics/bty350
[38]Póti, Á, et al.: Correlation of homologous recombination deficiency induced mutational signatures with sensitivity to parp inhibitors and cytotoxic agents. Genome Biology 20(240) (2019). doi:10.1186/s13059-019-1867-0
[39] Kasar, S., et al.: Whole-genome sequencing reveals activation-induced cytidine deaminase signatures during indolent chronic lymphocytic leukaemia evolution. Nature communications 6, 8866 (2015)
[40] Kim, J., et al.: Somatic ercc2 mutations are associated with a distinct genomic signature in urothelial tumors. Nature genetics 48(6), 600 (2016)
[41] Pavlidis, N., et al.: A mini review on cancer of unknown primary site: A clinical puzzle for the oncologists. Journal of Advanced Research 6, 375-382 (2015). doi:10.1016/j.jare.2014.11.007
[42] Jiao, W., et al.: A Deep Learning System Can Accurately Classify Primary and Metastatic Cancers Based on Patterns of Passenger Mutations. bioRxiv, 2017. 10.1101/214494
[43]Kübler, K., et al.: Tumor Mutational Landscape Is a Record of the Pre-malignant State. 10.1101/517565
[44] Rizvi, N. A., et al.: Mutational landscape determines sensitivity to PD-1 blockade in non-small cell lung cancer. Science 348(6230), 124-128 (2015). doi:10.1126/science.aaa1348
[45] Xu, Z., et al.: Assessment of tumor mutation burden calculation from gene panel sequencing data. OncoTargets and therapy 12, 3401-3409 (2019). doi:10.2147/OTT.S196638
[46] Trucco, L. D., et al.: Ultraviolet radiation-induced dna damage is prognostic for outcome in melanoma. Nature medicine 25(2), 221-224 (2019)

### REFERENCES FOR EXAMPLE 2

Alexandrov, L. B., Jones, P. H., Wedge, D. C., et al. 2015. Clock-like mutational processes in human somatic cells. Nat. Genet. 47, 1402-1407.
Alexandrov, L. B., Kim, J., Haradhvala, N. J., et al. 2020. The repertoire of mutational signatures in human cancer. Nature 578, 94-101.
Alexandrov, L. B., Nik-Zainal, S., Wedge, D. C., et al. 2013a. Deciphering signatures of mutational processes operative in human cancer. Cell Rep. 3, 246-259.
Alexandrov, L. B., Nik-Zainal, S., Wedge, D. C., et al. 2013b. Signatures of mutational processes in human cancer. Nature 500, 415-421.
Boutsidis, C., and Gallopoulos, E. 2008. Svd based initialization: A head start for nonnegative matrix factorization. Pattern Recognit. 41, 1350-1362.
Campbell, B. B., Light, N., Fabrizio, D., et al. 2017. Comprehensive analysis of hypermutation in human cancer. Cell 171, 1042-1056.
Cheng, D. T., Mitchell, T. N., Zehir, A., et al. 2015. Memorial Sloan Kettering-Integrated Mutation Profiling of Actionable Cancer Targets (MSK-IMPACT): A hybridization capture-based next-generation sequencing clinical assay for solid tumor molecular oncology. J. Mol. Diagn. 17,251-264.
Coffey, A. J., Kokocinski, F., Calafato, M. S., et al. 2011. The GENCODE exome: Sequencing the complete human exome. Eur. J. Hum. Genet. 19, 827-831.
Davies, H., Glodzik, D., Morganella, S., et al. 2017. HRDetect is a predictor of BRCA1 and BRCA2 deficiency based on mutational signatures. Nat. Med. 23, 517-525.
Farmer, H., McCabe, N., Lord, C. J., et al. 2005. Targeting the DNA repair defect in BRCA mutant cells as a therapeutic strategy. Nature 434, 917-921.
Frampton, G. M., Fichtenholtz, A., Otto, G. A., et al. 2013. Development and validation of a clinical cancer genomic profiling test based on massively parallel DNA sequencing. Nat. Biotechnol. 31, 1023-1031.
Garraway, L. A., and Lander, E. S. 2013. Lessons from the cancer genome. Cell 153, 17-37.
Gerstung, M., Jolly, C., Leshchiner, I., et al. 2020. The evolutionary history of 2,658 cancers. Nature 578, 122-128.
Gulhan, D. C., Lee, J. J., Melloni, G. E. M., et al. 2019. Detecting the mutational signature of homologous recombination deficiency in clinical samples. Nat. Genet. 51, 912-919.
Hanahan, D., and Weinberg, R. A. 2011. Hallmarks of cancer: The next generation. Cell 144, 646-674.
Haradhvala, N. J., Polak, P., Stojanov, P., et al. 2016. Mutational strand asymmetries in cancer genomes reveal mechanisms of DNA damage and repair. Cell 164, 538-549.
Helleday, T., Eshtad, S., and Nik-Zainal, S. 2014. Mechanisms underlying mutational signatures in human cancers. Nat. Rev. Genet. 15, 585-598.
Hoeck, A., Tjoonk, N. H., Boxtel, R. V., et al. 2019. Portrait of a cancer: Mutational signature analyses for cancer diagnostics. BMC Cancer 19, 457.
Huang, X., Wojtowicz, D., and Przytycka, T. M. 2018. Detecting presence of mutational signatures in cancer with confidence. Bioinformatics 34, 330-337.
Jiao, W., Atwal, G., Polak, P., et al. 2020. A deep learning system accurately classifies primary and metastatic cancers using passenger mutation patterns. Nat. Commun. 11, 728.
Kandoth, C., McLellan, M. D., Vandin, F., et al. 2013. Mutational landscape and significance across 12 major cancer types. Nature 502, 333-339.
Keilwagen, J., Grosse, I., and Grau, J. 2014. Area under precision-recall curves for weighted and unweighted data. PLOS One 9: e92209. www(dot)doi(dot)org/10(dot)1371/journal(dot)pone(dot)0092209.
Kim, J., Mouw, K. W., Polak, P., et al. 2016. Somatic ERCC2 mutations are associated with a distinct genomic signature in urothelial tumors. Nat. Genet. 48, 600-606.
Lawrence, M. S., Stojanov, P., Mermel, C. H., et al. 2014. Discovery and saturation analysis of cancer genes across 21 tumour types. Nature 505, 495-501.
Le, D. T., Durham, J. N., Smith, K. N., et al. 2017. Mismatch repair deficiency predicts response of solid tumors to PD-1 blockade. Science 357, 409-413. Lord, C. J., and Ashworth, A. 2017. PARP inhibitors: Synthetic lethality in the clinic. Science 355, 1152-1158. Morganella, S., Alexandrov, L. B., Glodzik, D., et al. 2016. The topography of mutational processes in breast cancer genomes. Nat. Commun. 7, 11383.
Nik-Zainal, S., Alexandrov, L. B., Wedge, D. C., et al. 2012. Mutational processes molding the genomes of 21 breast cancers. Cell 149, 979-993.
Nik-Zainal, S., Davies, H., Staaf, J., et al. 2016. Landscape of somatic mutations in 560 breast cancer whole-genome sequences. Nature 534, 47-54.
Nik-Zainal, S., Kucab, J. E., Morganella, S., et al. 2015. The genome as a record of environmental exposure. Mutagenesis 30, 763-770.
Nik-Zainal, S., Memari, Y., Davies, H. R. 2020. Holistic cancer genome profiling for every patient. Swiss Medical Weekly 150, w20158.
Perner, J., Abbas, S., Nowicki-Osuch, K., et al. 2020. The mutREAD method detects mutational signatures from low quantities of cancer DNA. Nat. Commun. 11, 3166.
Polak, P., Karlic′, R., Koren, A., et al. 2015. Cell-of-origin chromatin organization shapes the mutational landscape of cancer. Nature 518, 360-364.
Polak, P., Kim, J., Braunstein, L. Z., et al. 2017. A mutational signature reveals alterations underlying deficient homologous recombination repair in breast cancer. Nat. Genet. 49, 1476-1486.
R Core Team. 2017. R: A Language and Environment for Statistical Computing. R Foundation for Statistical Computing, Vienna, Austria.
Robin, X., Turck, N., Hainard, A., et al. 2011. pROC: An open-source package for R and S+ to analyze and compare ROC curves. BMC Bioinformatics 12, 77.
Rosenthal, R., McGranahan, N., Herrero, J., et al. 2016. deconstructSigs: Delineating mutational processes in single tumors distinguishes DNA repair deficiencies and patterns of carcinoma evolution. Genome Biol. 17, 31.
Sason, I., Chen, Y., Leiserson, M. D. M., et al. 2020. A mixture model for signature discovery from sparse mutation data, 271-272. In Proceedings of RECOMB 2020. Springer, Padua, Italy.
Staaf, J., Glodzik, D., Bosch, A., et al. 2019. Whole-genome sequencing of triple-negative breast cancers in a population-based clinical study. Nat. Med. 25, 1526-1533.
Stratton, M. R., Campbell, P. J., and Futreal, A. P. 2009. The cancer genome. Nature 458, 719.
Tate, J. G., Bamford, S., Jubb, H. C., et al. 2019. COSMIC: The catalogue of somatic mutations in cancer. Nucleic Acids Res. 47 (D1), D941-D947.
Temko, D., Tomlinson, I. P., Severini, S., et al. 2018. The effects of mutational processes and selection on driver mutations across cancer types. Nat. Commun. 9, 1857.
Tubbs, A., and Nussenzweig, A. 2017. Endogenous DNA damage as a source of genomic instability in cancer. Cell 168, 644-656.
Van Hoeck, A., Tjoonk, N. H., van Boxtel, R., et al. 2019. Portrait of a cancer: Mutational signature analyses for cancer diagnostics. BMC Cancer 19, 457.
Zugazagoitia, J., Guedes, C., Ponce, S., et al. 2016. Current challenges in cancer treatment. Clin. Ther. 38, 1551-1566.

What is claimed is:

1. A method of detecting mutational signatures of a sample in a collection of samples, each being characterized by nucleic acid sequencing information describing at least one mutation, the method comprising:
clustering said samples to provide clusters and respective exposure vectors, each exposure vector describing prior probabilities for a plurality of signatures to emit said mutation;
applying an optimization procedure to dynamically re-cluster said samples and to dynamically update said plurality of signatures and said exposure vectors; and
determining the mutational signatures in the sample and their exposure vector based on an output of said optimization procedure.

2. A method of detecting exposures of mutational signatures of a collection of samples, each being characterized by nucleic acid sequencing information describing at least one mutation, the method comprising:
receiving a collection of known mutational signatures;
clustering said samples to provide clusters and respective exposure vectors, each exposure vector describing prior probabilities for said signatures to emit said mutation;
applying an optimization procedure to dynamically re-cluster said samples and to dynamically update said exposure vectors; and
generating an output pertaining to said exposure vectors.

3. The method according to claim 1, wherein said clustering comprises calculating cluster prior probabilities, and wherein said optimization procedure dynamically updates said cluster prior probabilities.

4. The method according to claim 1, wherein said clustering comprises estimating mutational signatures shared among samples, and wherein said optimization procedure dynamically updates said shared mutational signatures.

5. The method according to claim 1, wherein said optimization procedure comprises an Expectation-Maximization procedure.

6. The method according to claim 1, wherein said optimization procedure comprises at least one of: a gradient descent procedure, a neural network procedure, an evolutionary procedure, and a simulated annealing procedure.

7. The method according to claim 1, wherein said signatures comprise mutational signatures of homologous recombination deficiencies.

8. The method according to claim 1, wherein said mutations comprise somatic mutations.

9. The method according to claim 1, wherein said mutations comprise cancer mutations.

10. The method according to claim 1, wherein for said sample and for at least a few samples in the collection, said nucleic acid sequencing information describes less than 20 mutations.

11. The method according to claim 1, wherein nucleic acid sequencing information comprises information obtained by targeted sequencing.

12. The method according to claim 1, further comprising obtaining said nucleic acid sequencing information by sequencing a collection of genome windows obtained from an output of a machine learning procedure trained to identify genome regions that are indicative of an activity of a signature of said at least one mutation.

13. The method according to claim 12, comprising feeding a whole genome to said trained machine learning procedure.

14. The method according to claim 12, wherein said trained machine learning procedure is trained to select an optimally-scoring subset of a set of genome windows.

15. The method according to claim 12, wherein said trained machine learning procedure is configured to divide said set into nonoverlapping windows, to compute, with a window scoring function, a measure of mutational signature activity in each window across said collection of samples, and to combine highest scoring windows.

16. The method according to claim 1, wherein at least one of said plurality of signatures comprises a set of values, each describing a probability associated with a known mutational category.

17. The method according to claim 16, wherein said known mutational category is one of a group of somatic mutation categories.

18. The method according to claim 16, wherein said known mutational category is one of a group of germline mutation categories.

19. A computer software product, comprising a non-transitory computer-readable medium in which program instructions are stored, which instructions, when read by a data processor, cause the data processor to receive nucleic acid sequencing information characterizing each sample in a collection of samples, to access a computer readable medium storing known mutational categories, and to execute the method according to claim 1.

20. A system for detecting mutational signatures of a sample in a collection of samples, the system comprising:
an input circuit receiving nucleic acid sequencing information characterizing each sample in the collection of samples;
a computer readable medium storing known mutational categories; and
a data processor configured for executing the method according to claim 1.
